(19) United States
(12) Patent Application Publication
(10) Pub. No.: US 2022/0172831 A1
(43) Pub. Date: Jun. 2, 2022

(54) MEDICAL CARE SUPPORT DEVICE

(71) Applicant: FUJIFILM Corporation, Tokyo (JP)

(72) Inventors: Tsuyoshi HIRAKAWA, Tokyo (JP); Hiroshi HIRAMATSU, Tokyo (JP); Jun AIZU, Tokyo (JP); Chuta KASAHARA, Tokyo (JP); Keiji TSUBOTA, Tokyo (JP); Misaki KAWAHARA, Tokyo (JP)

(73) Assignee: FUJIFILM Corporation, Tokyo (JP)

(21) Appl. No.: 17/673,791

(22) Filed: Feb. 17, 2022

Related U.S. Application Data
(63) Continuation of application No. PCT/JP2020/023878, filed on Jun. 18, 2020.

(30) Foreign Application Priority Data
Sep. 27, 2019 (JP) 2019-177973

(51) Int. Cl. G16H 40/20; G16H 10/60

(52) U.S. Cl. CPC G16H 40/20; G16H 10/60

(57) ABSTRACT

A medical care support device includes a display screen generation unit that generates a display screen for displaying a patient and a medical care process in association with each other, and an unread management unit that displays a status of the medical care process on the display screen. The unread management unit manages a viewer-specific status for each viewer who views the display screen separately from a main body status of the medical care process, changes the main body status by reflecting a status of a specific type of viewer in the main body status, and displays the viewer-specific status and the main body status in an identifiable manner on the display screen.

### CROSS-REFERENCE TO RELATED APPLICATIONS

This application is a Continuation of PCT International Application No. PCT/JP2020/023878 filed on Jun. 18, 2020, which claims priority under 35 U.S.C § 119(a) to Japanese Patent Application No. 2019-177973 filed on Sep. 27, 2019. Each of the above application(s) is hereby expressly incorporated by reference, in its entirety, into the present application.

### BACKGROUND OF THE INVENTION

### 1. Field of the Invention

The present invention relates to a medical care support device.

### 2. Description of the Related Art

In the medical field, integrated medical care support devices that share processes and results of medical care and the like between medical staff or between medical departments so that medical staff such as doctors and laboratory technicians can smoothly proceed with medical examinations, tests, and the like are being used. The medical care support device prevents oversight of information by displaying whether the shared information is read or unread in a discriminable manner, or the like (JP2016-189980A). Further, in JP2016-189980A, whether or not the information of “read” has been viewed by a designated reader is displayed in an identifiable manner.

### SUMMARY OF THE INVENTION

However, in JP2016-189980A, even in a case where the designated reader has not viewed the information, since the status is marked as read, there is a problem that it is overlooked that the designated reader has not viewed the information.

The present disclosure has been made in view of the above circumstance, and an object of the present disclosure is to provide a device that reduces oversight as described above.

According to an aspect of the present disclosure, there is provided a medical care support device comprising: a display screen generation unit that generates a display screen for displaying patient identification information and a medical care process in association with each other for each of a plurality of patients; an unread management unit that displays, on the display screen, a status including information indicating the medical care process being unread, read, or both of them, and manages a viewer-specific status separately from a main body status that is the status of the medical care process, the viewer-specific status being the status of each viewer who views the display screen; and a status change unit that changes the main body status by referring to the viewer-specific status and reflecting a status of a specific type of viewer among a plurality of types of viewers in the main body status. The unread management unit displays at least one of the viewer-specific status or the main body status in an identifiable manner on the display screen.

The unread management unit may display any of the viewer-specific status and the main body status in a selectable manner on the display screen.

The unread management unit may display both the viewer-specific status and the main body status on the display screen.

The viewer may be classified into a read target person and a non-read target person other than the read target person, the read target person may be classified into a management target person who is the specific type of viewer and a non-management target person other than the management target person, and in a case where the status of the management target person is changed from unread to read, the main body status may be changed from unread to read by reflecting the changed status.

In a case where a plurality of the management target persons are set and all the statuses of the plurality of management target persons are changed from unread to read, the main body status may be changed from unread to read by reflecting the changed statuses.

A correspondence table in which a first viewer who is allowed to deal with the medical care process alone and a second viewer who is prohibited from dealing with the medical care process alone are associated with each other may be referred to, and in a case where the second viewer is set as the management target person, the first viewer associated with the second viewer may be set as the management target person together with the second viewer.

The unread management unit may display the viewer-specific status of the read target person in an identifiable manner on the display screen.

The medical care support device may further comprise a setting change unit that changes settings of the viewer and the type of viewer.

The setting change unit may approve, in a case where the management target person is changed, a change to the management target person before the change, and change, in a case where the change is approved, the management target person.

In a case where the change is not approved, the viewer whose change is not approved may be set as the non-management target person.

The setting change unit may set a viewer of a request destination who has been requested to perform the medical care process as the read target person.

The medical care support device may further comprise a notification unit that receives an input of a comment to the request destination and notifies a designated request destination of the request and the comment.

The setting change unit may change the settings of the viewer and the type of viewer by displaying a correspondence list showing a correspondence between the medical care process and the read target person and reflecting a change operation performed on the correspondence list.

The medical care support device may further comprise a status transmission unit that transmits the viewer-specific status and the main body status to another medical care support device different from the medical care support device.

The display screen generation unit may transmit the display screen to another medical care support device different from the medical care support device.

With the medical care support device according to the aspect of the present disclosure, it is possible to reduce oversight of the fact that the person who should view does not view.

### BRIEF DESCRIPTION OF THE DRAWINGS

FIG. 1 is an explanatory diagram showing a configuration of a medical care support system.

FIG. 2 is an explanatory diagram showing a client terminal included in the medical care support system.

FIG. 3 is a block diagram showing a configuration of the client terminal.

FIG. 4 is a block diagram showing a function of the client terminal.

FIG. 5 is a block diagram showing a configuration of a medical care support device.

FIG. 6 is a block diagram showing a function of the medical care support device.

FIG. 7 is an explanatory diagram of a main body status management table.

FIG. 8 is an explanatory diagram of a viewer-specific status management table.

FIG. 9 is an initial screen.

FIG. 10 is a clinical flow screen.

FIG. 11 is a partially enlarged view of the clinical flow screen.

FIG. 12 is a partially enlarged view of the clinical flow screen.

FIG. 13 is an example of a badge indicating a status such as an unread status.

FIG. 14 is a timeline screen.

FIG. 15 is a layout screen.

FIG. 16 is an enlarged view of an endoscopic image field on the layout screen.

FIG. 17 is an unread data list screen.

FIG. 18 is a block diagram showing a configuration of the medical care support device.

FIG. 19 is an explanatory diagram showing a correspondence between a medical care process and a viewer-specific status.

FIG. 20 is an explanatory diagram showing a correspondence between a medical care process and a main body status.

FIG. 21 is an explanatory diagram of a correspondence table in which a medical care process and an authorized person are associated with each other.

FIG. 22 is a block diagram showing a configuration of the medical care support device.

FIG. 23 is a partially enlarged view of a viewer-specific status management table.

FIG. 24 is a partially enlarged view of a viewer-specific status management table.

FIG. 25 is a partially enlarged view of a viewer-specific status management table.

FIG. 26 is a block diagram showing a configuration of the medical care support device.

FIG. 27 is a block diagram showing a configuration of the medical care support device.

### DESCRIPTION OF THE PREFERRED EMBODIMENTS

### First Embodiment

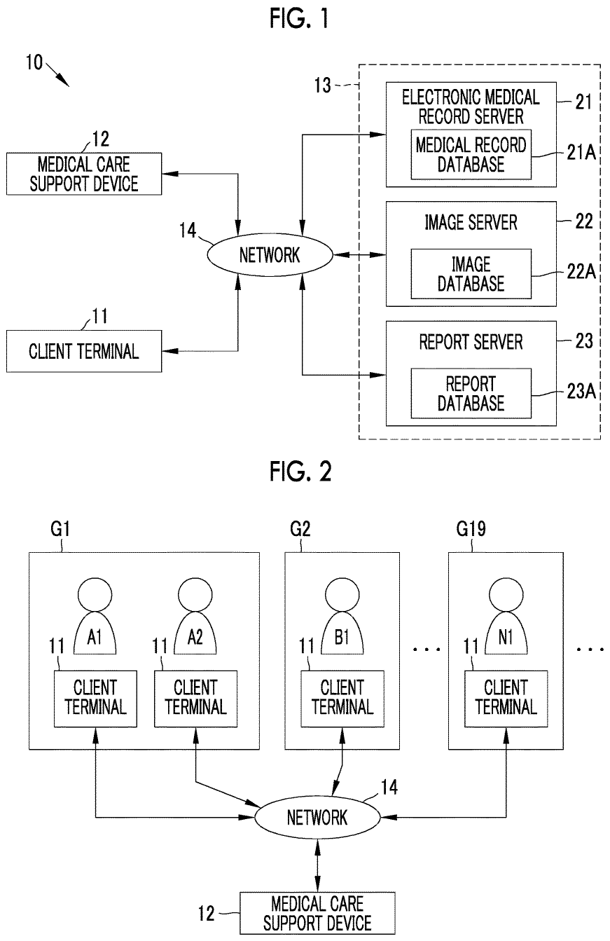

As shown in FIG. 1, a medical care support system 10 is a computer system that provides medical care support in a medical facility such as a hospital, and comprises a client terminal 11, a medical care support device 12, and a server group 13. These elements constituting the medical care support system 10 are connected to each other so as to be able to communicate with each other by using a network 14 such as a local area network (LAN) installed in the medical facility.

The client terminal 11 is a terminal for receiving a service (a function of the medical care support device 12) from the medical care support device 12, and is a computer directly operated by a medical staff such as a doctor, a laboratory technician, or a nurse (including the case of a tablet terminal, etc.), or the like. The client terminal 11 is installed in a medical department such as an internal medicine or a surgery, various examination departments such as a radiological examination department or a clinical examination department, a nurse center, or other necessary places. Further, the client terminal 11 can be provided for each medical staff, and can be shared by a plurality of medical staff. Therefore, as shown in FIG. 2, the medical care support system 10 includes a plurality of client terminals 11. For example, a group G1 is an “internal medicine” to which a doctor A1 and a doctor A2 belong, and the doctor A1 and the doctor A2 each have a client terminal 11. Similarly, for example, a group G2 is a “surgery” to which a doctor B1 belongs, and the group G2 has at least one client terminal 11. Further, for example, a group G19 is a “radiology department” to which a technician N1 belongs, and the group G19 has at least one client terminal 11.

The medical care support device 12 provides the client terminal 11 with a display screen including medical care data (for example, an image or the like itself) and/or information indicating the location of the medical care data (for example, a link to an image or the like), for example, in response to a request from the client terminal 11. Medical care data is images, reports, examination results, and other medical care processes acquired or created in medical examinations, tests, surgeries, and the like, or is data obtained as a result of medical care or information indicating the location of these (so-called links (aliases), etc.). The medical care support device 12 acquires medical care data to be used on the display screen from the server group 13.

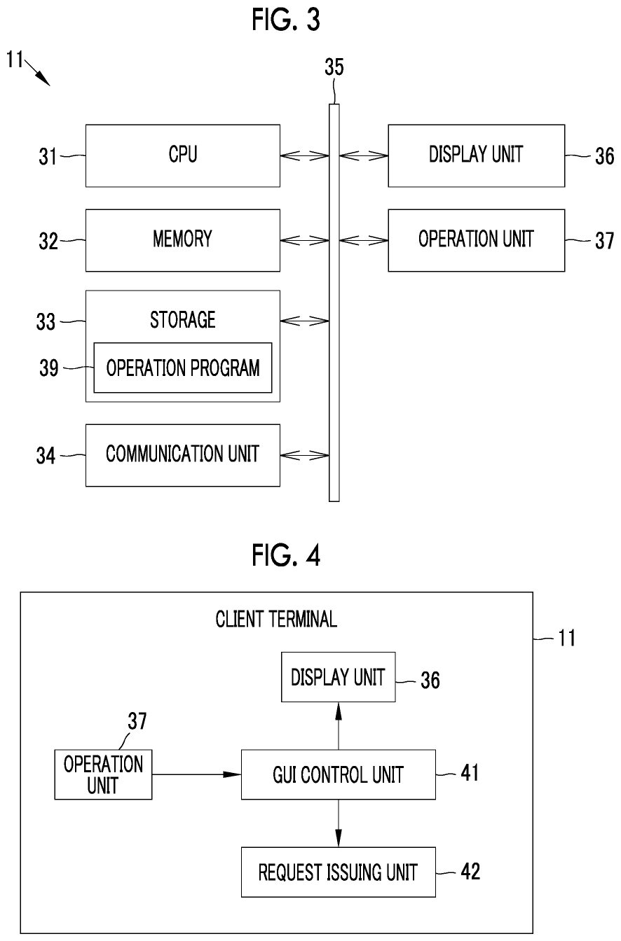

A display screen provided by the medical care support device 12 to the client terminal 11 refers to data used by the client terminal 11 to form a screen of a display unit 36 (see FIG. 3) of the client terminal 11. Further, the display screen provided by the medical care support device 12 to the client terminal 11 includes not only data for full-screen display that the client terminal 11 constitutes the display of the entire screen but also data that constitutes the display related to a part of the screen. For example, in the present embodiment, the medical care support device 12 provides the client terminal 11 with a display screen that can be displayed in a general window format on a part of the screen of the display unit 36.

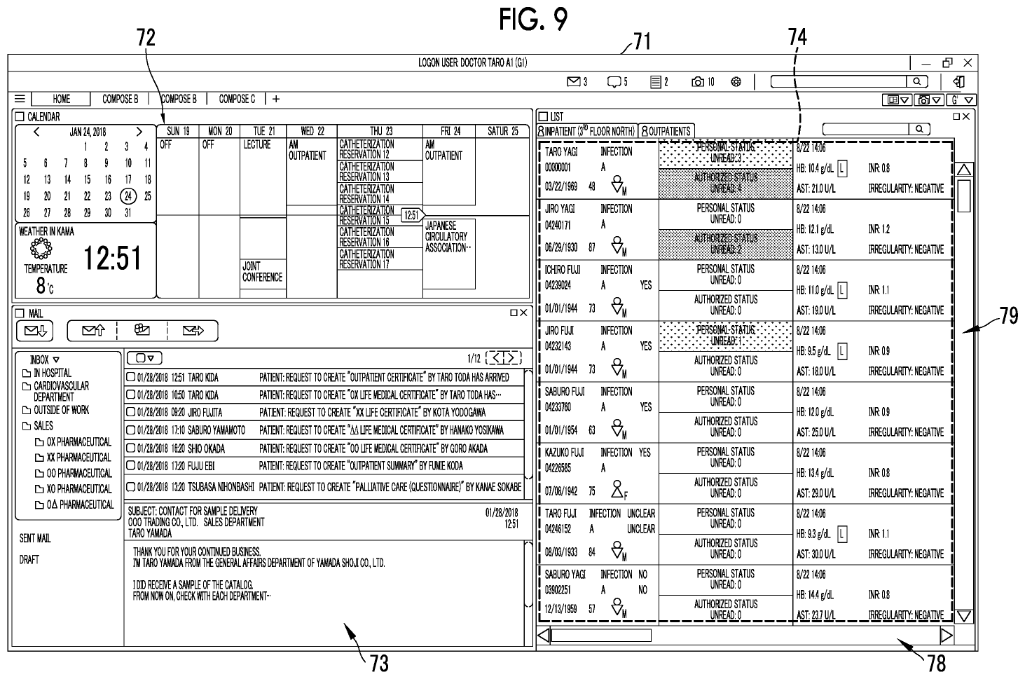

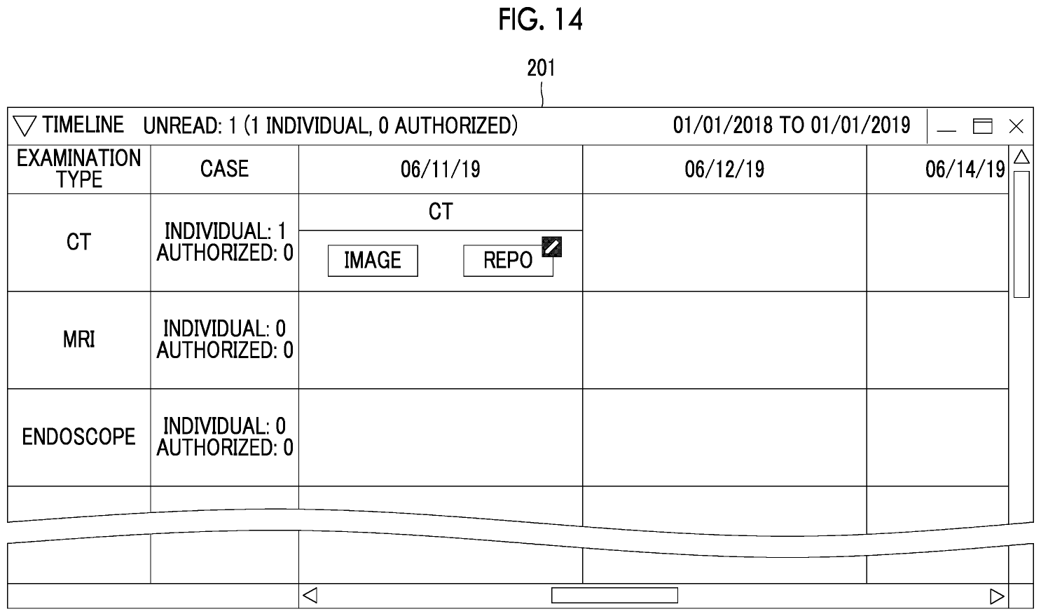

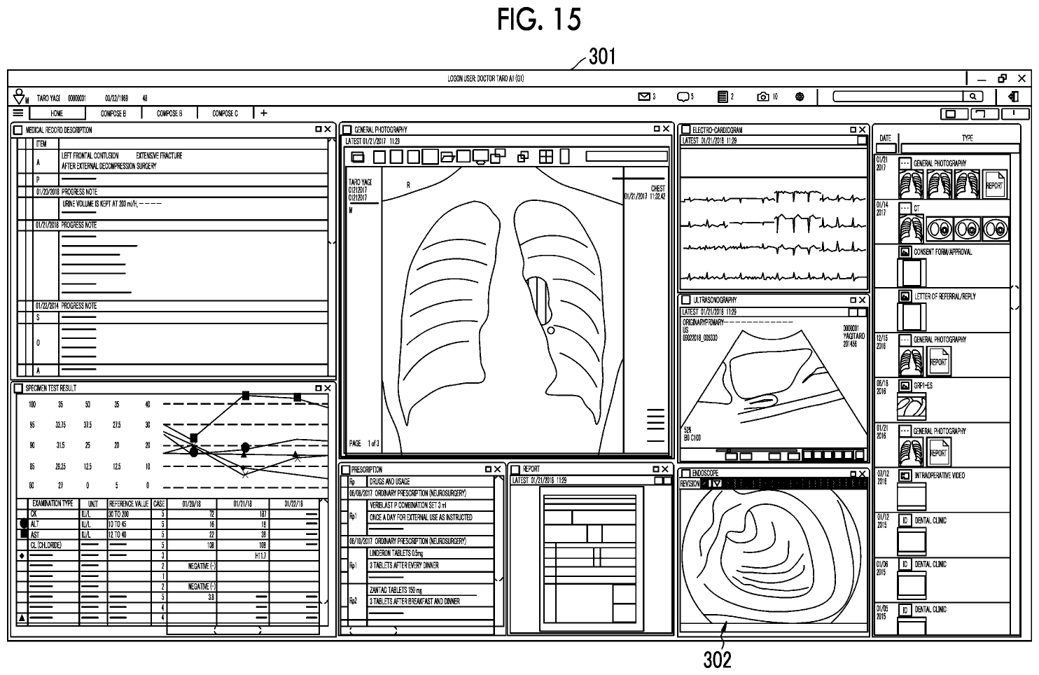

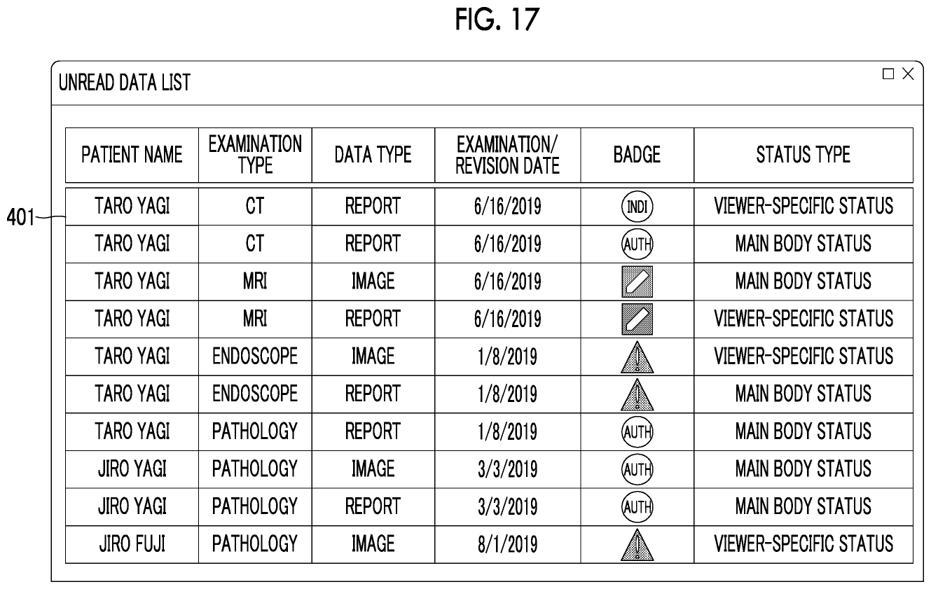

Specifically, the display screens provided by the medical care support device 12 to the client terminal 11 include a clinical flow screen 81 (see FIG. 9), a timeline screen 201 (see FIG. 14), a layout screen 301 (see FIG. 15), an unread data list screen 401 (see FIG. 17), and the like. The clinical flow screen 81 is a display screen for displaying patient identification information and a part or all of the medical care process in association with each other for each of a plurality of patients. Patient identification information is, for example, identification data (ID) such as the patient's name, date of birth, age, or gender, or a unique number and/or symbol given to the patient (hereinafter referred to as a patient ID). A medical care process refers to the process or result of medical care that has already been performed and that is scheduled to be performed in the future. Therefore, the medical care process includes various medical care data obtained in the process of medical care or as a result of medical care (for example, data such as a medical examination record, a result of a specimen test, a vital sign, an order for examinations, a treatment record, an examination image, a report, or findings). In addition, the medical care process may include not only medical care data that has already been acquired, but also medical care data that is scheduled to be acquired. The medical care data that is scheduled to be acquired is, for example, information regarding the presence or absence of an order for a specific examination, a scheduled date and time thereof, the type of medical care data that is scheduled to be acquired, and the like. Further, in the present embodiment, in a case where one medical care process includes a plurality of items (items such as examination results), the term “medical care process” refers to any of the items that constitute the medical care process, not the entire medical care process (a collection of a plurality of items). The timeline screen 201 is a display screen for displaying a part or all of the medical care process of a specific patient on one screen in a time series. The layout screen 301 is a display screen for displaying a part or all of the medical care process of a specific patient by arranging them vertically and horizontally (for example, arranging them in a tile shape). The unread data list screen 401 is a display screen for displaying a list of medical care data whose contents have not been confirmed by medical staff and/or medical care data to be acquired in the future.

The medical care support device 12 provides a display screen to the client terminal 11 in a description format using a markup language such as extensible markup language (XML) data, for example. The client terminal 11 displays an XML format display screen using a web browser. The medical care support device 12 can provide a display screen to the client terminal 11 in another format such as JavaScript (registered trademark) object notation (JSON) instead of XML.

The server group 13 searches for medical care data in response to the request from the medical care support device 12, and provides the medical care data corresponding to the request to the medical care support device 12. The server group 13 includes an electronic medical record server 21, an image server 22, a report server 23, and the like.

The electronic medical record server 21 has a medical record database 21A for storing electronic medical records. An electronic medical record is a collection of one or a plurality of pieces of medical care data. Specifically, the electronic medical record includes, for example, medical care data such as a medical examination record, a result of a specimen test, a patient's vital sign, an order for examinations, a treatment record, or accounting data. The electronic medical record can be input and viewed using the client terminal 11.

A medical examination record is a record of the contents and results of the interview or palpation, the disease name, or the like. A specimen is blood or tissue collected from a patient, or the like, and a specimen test is a blood test, a biochemical test, or the like. A vital sign is data indicating a patient's condition such as a patient's pulse, blood pressure, or body temperature. An order for examinations is a request for examinations such as a specimen test, photography using various modality, report creation, treatment or surgery, medication, or the like. A treatment record is a record of treatment, surgery, medication, prescription, or the like. Accounting data is data related to consultation fees, drug fees, hospitalization fees, and the like.

The image server 22 is a so-called PACS (picture archiving and communication system) server, and has an image database 22A in which examination images are stored. An examination image is an image obtained by various image examinations such as computed tomography (CT) examination, magnetic resonance imaging (MRI) examination, X-ray examination, ultrasonography, and endoscopy. These examination images are recorded in a format conforming to, for example, the digital imaging and communications in medicine (DICOM) standard. The examination image can be viewed using the client terminal 11.

The report server 23 has a report database 23A for storing an interpretation report. An interpretation report (hereinafter simply referred to as a report) is a report that summarizes the interpretation results of the examination image obtained by the image examination. The interpretation of the examination image is performed by a radiologist. The report can be created and/or viewed using the client terminal 11.

A patient ID is attached to each of the above electronic medical records, examination images, and reports. In addition to the patient ID, information that identifies the medical staff who input the medical care data for each medical care data is attached to the electronic medical record. In addition to the patient ID, information that identifies the medical staff (specifically, the laboratory technician) who performed the examination is attached to the examination image. Information that identifies the medical staff (specifically, the radiologist) that created the report is attached to the report. Information that identifies the medical staff is an ID such as the name of the medical staff or a unique number and/or symbol given to each medical staff (hereinafter referred to as a medical staff ID).

The client terminal 11, the medical care support device 12, and the servers 21 to 23 constituting the server group 13 are configured by installing an operating system program and an application program such as a server program or a client program based on a computer such as a server computer, a personal computer, or a workstation. That is, the basic configurations of the client terminal 11, the medical care support device 12, and the servers 21 to 23 constituting the server group 13 are the same, and a central processing unit (CPU), a memory, a storage, a communication unit, etc., and a connection circuit for connecting these are provided. The communication unit is a communication interface (LAN port or the like) for connecting to the network 14. The connection circuit is, for example, a motherboard that provides a system bus and/or a data bus and the like.

As shown in FIG. 3, the client terminal 11 comprises a display unit 36 and an operation unit 37 in addition to a CPU 31, a memory 32, a storage 33, a communication unit 34, and a connection circuit 35. The display unit 36 is, for example, a display using a liquid crystal display or the like, and has at least a screen for displaying a display screen provided by the medical care support device 12. The operation unit 37 is, for example, a pointing device such as a mouse and/or an input device such as a keyboard. The display unit 36 and the operation unit 37 can form a so-called touch panel.

The client terminal 11 stores an operation program 39 in addition to the operating system program and the like in the storage 33. The operation program 39 is an application program for receiving the function of the medical care support device 12 by using the client terminal 11. In the present embodiment, the operation program 39 is a web browser program. Here, the operation program 39 can be a dedicated application program for receiving the function of the medical care support device 12. The operation program 39 may include one or a plurality of gadget engines for controlling a part or all of the display screen provided by the medical care support device 12. A gadget engine is a subprogram that exhibits various functions by operating alongside a web browser or the like.

In a case where the operation program 39 is activated in the client terminal 11, as shown in FIG. 4, the CPU 31 of the client terminal 11 functions as a graphical user interface (GUI) control unit 41 and a request issuing unit 42 in cooperation with the memory 32.

The GUI control unit 41 displays the display screen provided by the medical care support device 12 on the web browser in the display unit 36. The GUI control unit 41 controls the client terminal 11 in response to an operation instruction input using the operation unit 37, such as a button click operation with a pointer.

The request issuing unit 42 issues various processing requests (hereinafter referred to as processing requests) to the medical care support device 12 in response to the operation instruction of the operation unit 37. The processing request issued by the request issuing unit 42 is, for example, a distribution request for the display screen, an edit request for the display screen, or the like. The request issuing unit 42 transmits the processing request to the medical care support device 12 via the communication unit 34 and the network 14.

The distribution request for the display screen is for requesting the medical care support device 12 to distribute a display screen having a specific configuration. For example, the distribution can be received by designating any one of the clinical flow screen 81, the timeline screen 201, the layout screen 301, the unread data list screen 401, and the like, depending on the distribution request for the display screen.

The edit request for the display screen is for requesting the medical care support device 12 to edit the contents of the medical care data and the like to be displayed on the display screen after receiving the distribution of the display screen having a specific configuration from the medical care support device 12. For example, in a case where the distribution of the clinical flow screen 81 is received, the edit request for the display screen is a request for designating or changing a list of patients to be displayed, designating or changing a display target period of the medical care process, designating or changing the medical care process to be displayed, or sorting the display contents.

The distribution request and/or edit request for the display screen includes information such as a medical staff ID and an address on the network of the client terminal 11. The medical staff ID is entered on a login screen (not shown) for the medical care support system 10 (or the medical care support device 12).

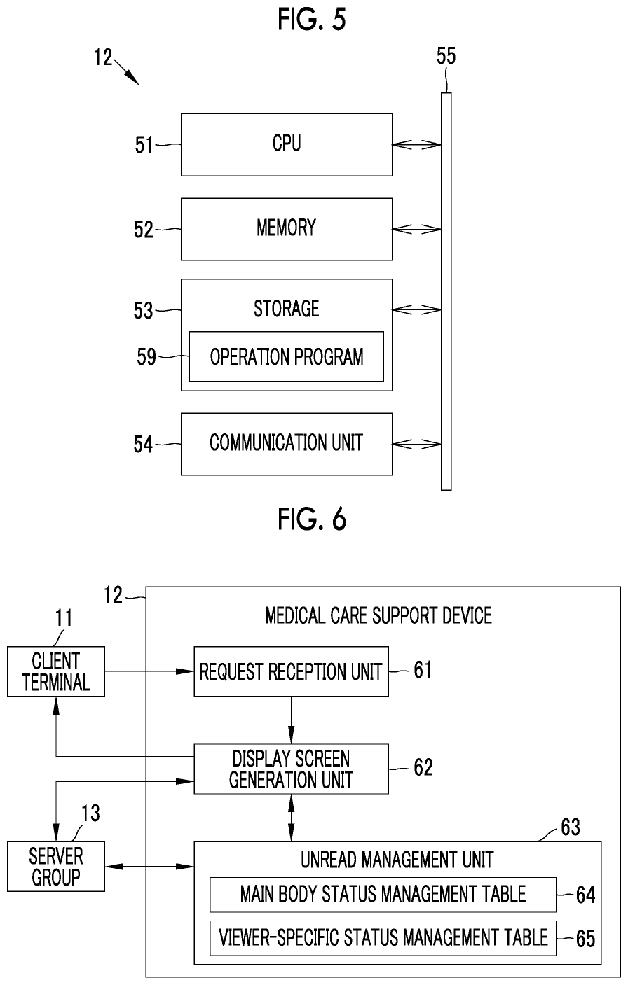

As shown in FIG. 5, the medical care support device 12 comprises a CPU 51, a memory 52, a storage 53, a communication unit 54, and a connection circuit 55. The medical care support device 12 may comprise a display unit and/or an operation unit in the same manner as the client terminal 11 as necessary. In addition, a display unit and/or an operation unit can be attached as necessary. However, in the present embodiment, the medical care support device 12 does not have a display unit and an operation unit.

The medical care support device 12 stores an operation program 59 in addition to the operating system and the like in the storage 53. The operation program 59 is an application program for causing the computer constituting the medical care support device 12 to function as the medical care support device 12. In a case where the operation program 59 is activated, as shown in FIG. 6, the CPU 51 of the medical care support device 12 functions as a request reception unit 61, a display screen generation unit 62, an unread management unit 63 (status change unit), and the like in cooperation with the memory 52.

The request reception unit 61 receives various processing requests such as a distribution request and an edit request for the display screen from the client terminal 11. In a case where the request reception unit 61 receives various processing requests, the request reception unit 61 inputs a processing instruction to each unit that executes the corresponding processing according to the content of the requested processing. For example, in a case where there is a distribution request for the display screen from the client terminal 11, the request reception unit 61 inputs a generation instruction of the corresponding display screen to the display screen generation unit 62. Similarly, in a case where there is an edit request for the display screen from the client terminal 11, the request reception unit 61 inputs an edit instruction of the corresponding display screen to the display screen generation unit 62. The request reception unit 61 also receives a request to log in to the medical care support device 12, and a login processing unit (not shown) executes login processing such as confirmation of the medical staff ID and password.

The display screen generation unit 62 generates or edits various display screens such as the clinical flow screen 81. In the present embodiment, in a case where there is a new distribution request for the display screen, the display screen generation unit 62 generates XML data representing the display screen, and in a case where there is an edit request for the display screen, the display screen generation unit 62 edits the XML data created earlier according to the request content. The display screen generation unit 62 accesses the server group 13 as necessary, and acquires information regarding a medical care process or the like used for generating or editing the display screen. The display screen generation unit 62 can hold a part or all of the information regarding the medical care process or the like acquired from the server group 13 in order to reduce the access frequency to the server group 13. In a case where the login processing unit normally completes the login processing, the display screen generation unit 62 generates an initial screen 71 (see FIG. 9) to be displayed first after login. Further, in the case of creating or editing the initial screen 71, the display screen generation unit 62 acquires the information necessary for generating or editing the initial screen 71 from the server group 13, the client terminal 11, or another device or system that is linked with the medical care support system 10.

The unread management unit 63 manages a status of a medical care process included in the display screen generated or edited by the display screen generation unit 62. A status of a medical care process is management information including information indicating the medical care process being unread, read, or both of them. Unread (management information including being unread) refers to a state in which the medical staff has not yet performed predetermined processing to be performed, such as viewing, editing, inserting comments, and approval processing (hereinafter also referred to as viewing or the like) for the medical care process. Read (management information including being read) refers to a state in which the medical staff has already performed predetermined processing to be performed, such as viewing or the like for the medical care process. Management information including information of both “unread” and “read” is substantially management information of “unread” or “read”. This is because unread and read represent alternative states, so that unread represents not read at the same time, and read represents not unread at the same time.

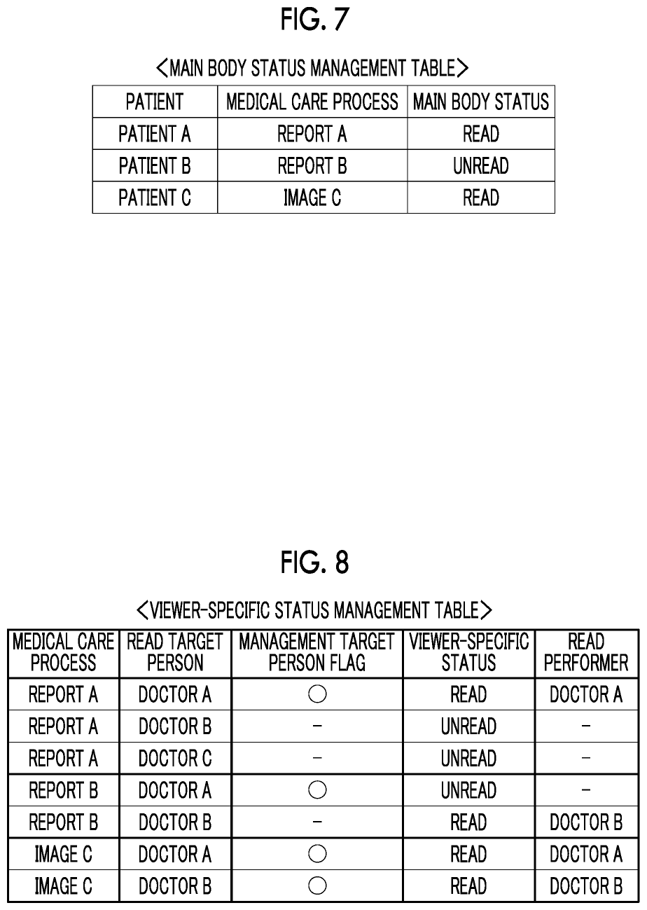

The unread management unit 63 is provided with a main body status management table 64 and a viewer-specific status management table 65. As shown in FIG. 7, the main body status management table 64 is provided to manage a status of the medical care process itself (hereinafter referred to as a main body status), and in the example of FIG. 7, a patient, a medical care process, and a main body status are associated with each other. The patient indicates a patient who is the target of the medical care process, and in the example of FIG. 7, a patient A, a patient B, and a patient C are registered. Further, in the example of FIG. 7, a report A is registered as the medical care process of the patient A, a report B is registered as the medical care process of the patient B, and an image C is registered as the medical care process of the patient C. Then, in the example of FIG. 7, the main body status of the report A is registered as read, the main body status of the report B is registered as unread, and the main body status of the image C is registered as read.

As will be described later, the main body status can be derived from the viewer-specific status managed by the viewer-specific status management table 65. Therefore, in the example of FIG. 7, the main body status field is provided in the main body status management table 64, and the main body status is registered here. However, the main body status field may be abolished, and the main body status may be derived from the viewer-specific status by referring to the viewer-specific status management table 65.

As shown in FIG. 8, the viewer-specific status management table 65 is provided to manage a status of each viewer (hereinafter referred to as a viewer-specific status) for each medical care process. In the example of FIG. 8, a medical care process, a read target person, a management target person flag, a viewer-specific status, and a read performer are associated with each other. In the example of FIG. 8, the above-mentioned report A, report B, and image C are registered as the medical care process.

The read target person and the management target person flag are for identifying the type of viewer. Viewers are classified into read target persons who are allowed to view the medical care process (having viewing authority) and non-read target persons other than the read target persons. Further, the read target persons are classified into a management target person, which is a specific type of viewer of the present disclosure, in which the viewer-specific status is reflected in the main body status, and a non-management target person other than the management target person. In FIG. 8, a viewer registered in the read target person field is a read target person, and a read target person with an indicator of “o” in the management target person flag field is a management target person. Specifically, the read target persons of the report A are a doctor A, a doctor B, and a doctor C, the read target persons of the report B are the doctor A and the doctor B, and the read target persons of the image C are the doctor A and the doctor B, the management target person of the report A is the doctor A, the management target person of the report B is the doctor A, and the management target persons of the image C are the doctor A and the doctor B.

The classifying of viewers, that is, whether each viewer is classified into any of a management target person (read target person), a non-management target person (read target person), and a non-read target person, can be set and changed as appropriate. In addition, the medical care support device 12 and the like may be configured to be automatically set and changed according to the type of medical care process and the like. However, it is preferable to set persons who are currently involved in the medical care process or who have been involved in the past as read target persons. Further, among such read target persons, it is preferable to set a person who is in a position to lead or supervise the medical care process (for example, an attending physician, a doctor in charge, a radiologist in a case where the medical care process is interpretation, a surgeon in a case where the medical care process is surgery, or the like) as a management target person.

Then, in the example of FIG. 8, regarding the report A, the viewer-specific status of the doctor A is marked as read, and the viewer-specific status of the doctor B and the doctor C is marked as unread. Regarding the report B, the viewer-specific status of the doctor A is marked as unread, and the viewer-specific status of the doctor B is marked as read. Further, regarding the image C, the viewer-specific status of the doctor A and the doctor B is marked as read.

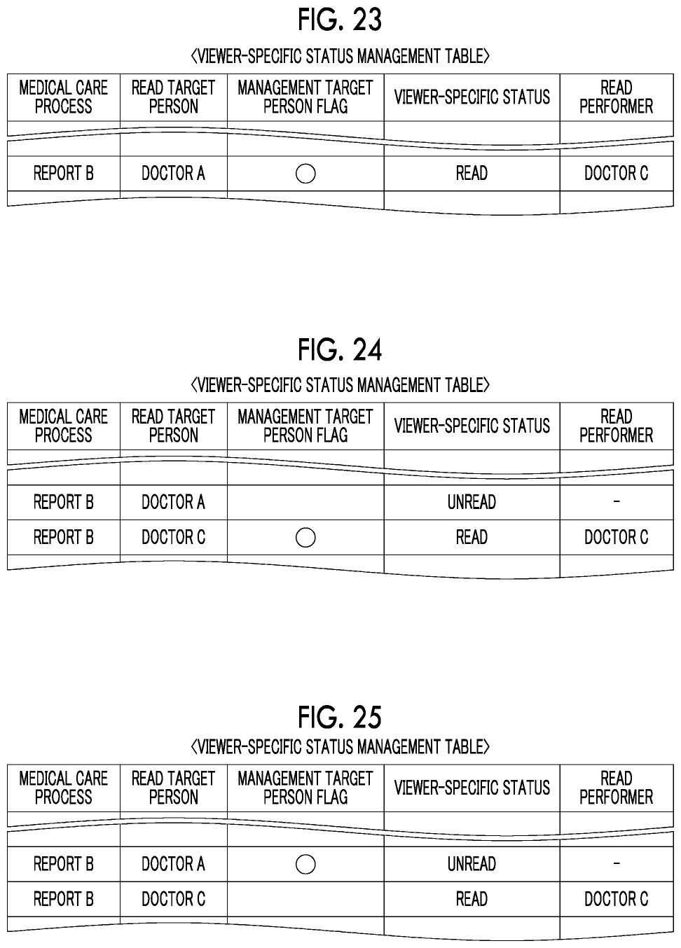

In a case where the viewer-specific status is read, the read performer is information indicating which viewer's viewing or the like causes the viewer-specific status to be read. In the example of FIG. 8, the read target person and the read performer match, but the read performer may be different from the read target person (see FIG. 23). That is, the own viewer-specific status may be marked as read by another person's viewing or the like. However, in this case, it is preferable that the viewer-specific status of this viewer can be changed to read by another person only in a case where the request or approval of the viewer whose viewer-specific status is changed to read is received. Of course, the own viewer-specific status may be changed from read to unread by the operation or the like of another person. Also in this case, only in a case where the request or approval of the viewer whose viewer-specific status is changed is received, it is preferable that the viewer-specific status of this viewer can be changed to unread by another person.

Referring back to FIG. 6, the unread management unit 63 acquires the viewer-specific status, that is, information indicating which medical care process each viewer has viewed, or the like, for example, from a device or system that is linked with the medical care support device 12, such as the server group 13. In addition, the unread management unit 63 can directly acquire the viewer-specific status from the client terminal 11 by acquiring the operation information of the client terminal 11. The unread management unit 63 monitors the viewer-specific status by acquiring the viewer-specific status in real time or periodically, and in a case where the viewer-specific status of the read target person in the medical care process is changed, the unread management unit 63 updates the viewer-specific status management table 65 by reflecting the changed contents.

Further, in a case where the viewer-specific status of the management target person is changed by updating the viewer-specific status management table 65, the unread management unit 63 updates the main body status management table 64 by reflecting this change in the main body status. Specifically, in a case where the management target person views the medical care process and the viewer-specific status of the medical care process is marked as read, the main body status is changed to read by reflecting the status in the main body status. In a case where there are a plurality of management target persons for one medical care process, in the present embodiment, the main body status is marked as read in a case where all the viewer-specific statuses of the management target persons is marked as read. However, for example, in a case where the viewer-specific status of some of the management target persons is marked as read, such as a case where the viewer-specific status of one of the management target persons or a predetermined percentage (for example, more than half) of the management target persons is marked as read, the main body status may be marked as read.

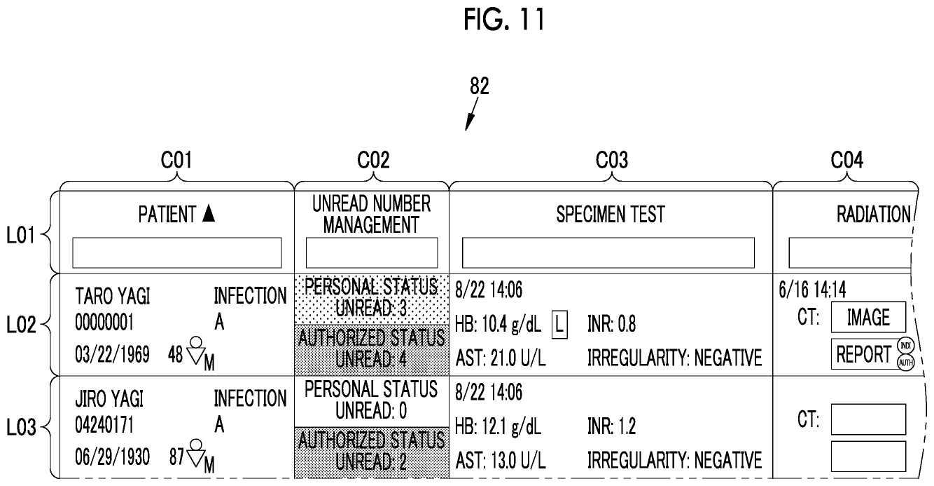

In this way, the unread management unit 63 manages the main body status, which is the status of the medical care process itself, and the viewer-specific status, which is the status of each viewer for each medical care process, separately. Then, the viewer-specific status of a specific type of viewer (in the present embodiment, a management target person) is reflected in the main body status to change the main body status. That is, the unread management unit 63 manages the main body status and the viewer-specific status separately. Further, the unread management unit 63 corresponds to a status change unit of the present disclosure, which changes the main body status by reflecting the viewer-specific status of a specific type of viewer in the main body status. Then, the unread management unit 63 displays the main body status and the viewer-specific status in an identifiable manner on the display screen generated or edited by the display screen generation unit 62. Specifically, regarding the main body status and the viewer-specific status, the content (whether it is marked as read or unread) and the type (whether it is the main body status or the viewer-specific status) are displayed in an identifiable manner (see FIGS. 11 to 13).

In this way, by managing the main body status and the viewer-specific status separately and displaying them in an identifiable manner, it is possible to reduce the oversight of the medical care process by the medical staff. That is, in a case where the main body status and the viewer-specific status cannot be identified without managing them separately, even in a case where it is viewed by a less relevant viewer (in the present embodiment, a viewer other than the management target person) who is not directly involved in the medical care process, the medical care process is treated as “read”. As a result, a highly relevant viewer (in the present embodiment, a management target person) who is directly involved in the medical care process may overlook that this medical care process is unread. However, such oversight can be reduced by managing the main body status and the viewer-specific status separately and displaying them in an identifiable manner. In addition to the viewer-specific status, which is the viewer's own status, the main body status, which is the status of the medical care process itself, can be confirmed. Therefore, even a viewer who is less related to the medical care process can easily find that a viewer who is highly related to the medical care process is unread by confirming the main body status, which can contribute to the reduction of oversight.

A specific display mode that makes it possible to distinguish between the main body status and the viewer-specific status can be appropriately set. For example, in the display screen generated or edited by the display screen generation unit 62, the unread management unit 63 can attach a predetermined mark (for example, a predetermined form of “badge”) indicating the main body status and the viewer-specific status to the display portion of the target medical care process. That is, the unread management unit 63 can display the content and type of main body status and the viewer-specific status in an identifiable manner on the display screen depending on the presence or absence or form of the specific mark attached to the medical care process. The mark display is easy to understand visually, and it is especially easy to achieve the purpose of reducing oversight. The form of the mark is the shape (including the difference in size), color, pattern, etc. of the mark, or a combination thereof. In addition, the content and type of main body status and the viewer-specific status may be indicated by the form of one mark, the content and type of main body status and the viewer-specific status may be indicated by the presence or absence or form of a plurality of marks such as indicating the content of the main body status by the presence or absence or form of a first mark, and indicating the content of the viewer-specific status by the presence or absence or form of a second mark. Of course, the content and type of main body status and the viewer-specific status may be displayed in an identifiable manner by displaying text, that is, by character notation.

In the present embodiment, the display screen generation unit 62 generates or edits XML data representing the display screen. Therefore, the unread management unit 63 edits the XML data generated or edited by the display screen generation unit 62, or causes the display screen generation unit 62 to edit the data (including the case of editing at the same time as the display screen is generated), so that the main body status and the viewer-specific status are displayed in an identifiable manner on the display screen generated by the display screen generation unit 62. Specifically, the unread management unit 63 designates a medical care process with a mark, the color and form of the mark to be attached, or designates a medical care process (or a medical care process without a mark) that removes the already attached mark in a case of editing the XML data or in a case where the display screen generation unit 62 is caused to edit the XML data. By editing the XML data according to this designation, the main body status and the viewer-specific status are displayed in an identifiable manner on the display screen generated by the display screen generation unit 62.

The display screen generation unit 62 distributes the display screen generated or edited as described above to the client terminal 11 via the communication unit 54. Thereby, the client terminal 11 displays the display screen provided by the display screen generation unit 62 on the display unit 36. In a case where the unread management unit 63 further edits the display screen generated or edited by the display screen generation unit 62 in order for the unread management unit 63 to display the above-mentioned mark on the display screen, the unread management unit 63 can provide the display screen to the client terminal 11.

In addition, the unread management unit 63 manages the number of each status of the main body status and the viewer-specific status displayed on the display screen, specifically, the number of unread and/or read items. The unread management unit 63 can display the number of one or both of each status on the display screen as necessary, and in the present embodiment, both the number of the main body status and the number of the viewer-specific status are displayed (see FIG. 11).

The unread management unit 63 can manage the status of “revised” and/or “important” in addition to the above-mentioned unread, read, or both as the status of the medical care process. Furthermore, regarding the “revised” and/or “important” status, the main body status and the viewer-specific status can be managed separately in the same manner as the unread, read, or both statuses described above, and the information under these management can be displayed on the display screen in an identifiable manner. Here, “revised” is a status indicating that the content of the medical care process that was unread or read has been changed. For example, revision of an examination image refers to adding a newly acquired examination image to an already acquired examination image, replacing a part or all of the already acquired examination image with a newly acquired examination image, or the like. Revision of a report refers to adding information such as findings to an existing report, changing the contents of an existing report, or the like. Further, “important” is a status indicating a medical care process in which the medical staff has set to call attention to the medical care process to himself/herself or other medical staff (for example, setting an important flag).

In a case where there has been a “revision” in the medical care process and/or in a case where an “important” setting has been made in the medical care process, the unread management unit 63 can display that there has been a “revision” and/or that an “important” setting has been made in an identifiable manner by attaching a predetermined mark to the display portion of the medical care process. In addition, the unread management unit 63 can display each case of a case where there is a “revision” and the main body status is read, a case where there is a “revision” and the main body status is unread, a case where there is a “revision” and the viewer-specific status is read, a case where there is a “revision” and the viewer-specific status is unread, a case where an “important” setting is made and the main body status is read, a case where an “important setting is made and the main body status is unread, a case where an “important setting is made and the viewer-specific status is read, and a case where an “important setting is made and the viewer-specific status is unread, in an identifiable manner by changing the presence or absence of the mark or the form. In addition, the unread management unit 63 may change the status of “read” to “unread” for the main body status and the viewer-specific status of the medical care process for which there has been a “revision” and/or an “important” setting has been made.

In addition, the unread management unit 63 can manage each status of the main body status and the viewer-specific status separately for each requester of the medical care process, and can display each status thereof on the display screen in a display mode that can be distinguished for each requester. The distinction for each requester of the medical care process includes not only distinguishing each medical staff but also distinguishing each group (medical department, etc.) to which the medical staff belongs. Therefore, for example, the requester of the medical care process may be distinguished into three types of “self”, “self-dept”, and “other depts”, and regarding one or both of the main body status and the viewer-specific status, whether the requester of the medical care process belongs to any of the three types of “self”, “self-dept”, and “other depts” may be displayed in an identifiable manner. Here, “self” represents a case where the requester of the medical care process is the viewer himself/herself. “Self-dept” represents a case where the requester of the medical care process belongs to a group to which the viewer himself/herself belongs and is a medical staff other than the viewer himself/herself “Other depts” represent a case where the requester of the medical care process is a medical staff who belongs to a group to which the viewer himself/herself does not belong.

By doing so, in a case where the viewer is a medical staff, the viewer can clearly recognize the medical care process that he/she should view, in distinction from others. As a result, the viewer can reduce the oversight of the medical care process that he/she should view. In addition, the viewer can clearly recognize the medical care process that the medical staff of his/her department, including himself/herself, should view, in distinction from others. As a result, the viewer can efficiently support or manage the tasks of the group's medical staff and reduce oversight of the medical care process on a group-by-group basis. In addition, the viewer can help reduce oversight by the medical staff in other departments by contacting other departments as necessary. Furthermore, in a case where the viewer is the manager (responsible person) of a medical facility such as a hospital, the facility as a whole can be managed so that the medical care process is not overlooked.

The medical care support system 10 having the above configuration operates as follows. First, in a case where the medical staff logs in to the medical care support system 10 using the client terminal 11, the display screen generation unit 62 generates the initial screen 71 shown in FIG. 9 based on the settings and the like set for each medical staff, and provides the initial screen 71 to the client terminal 11. Thereby, the client terminal 11 displays the initial screen 71 on the screen of the display unit 36.

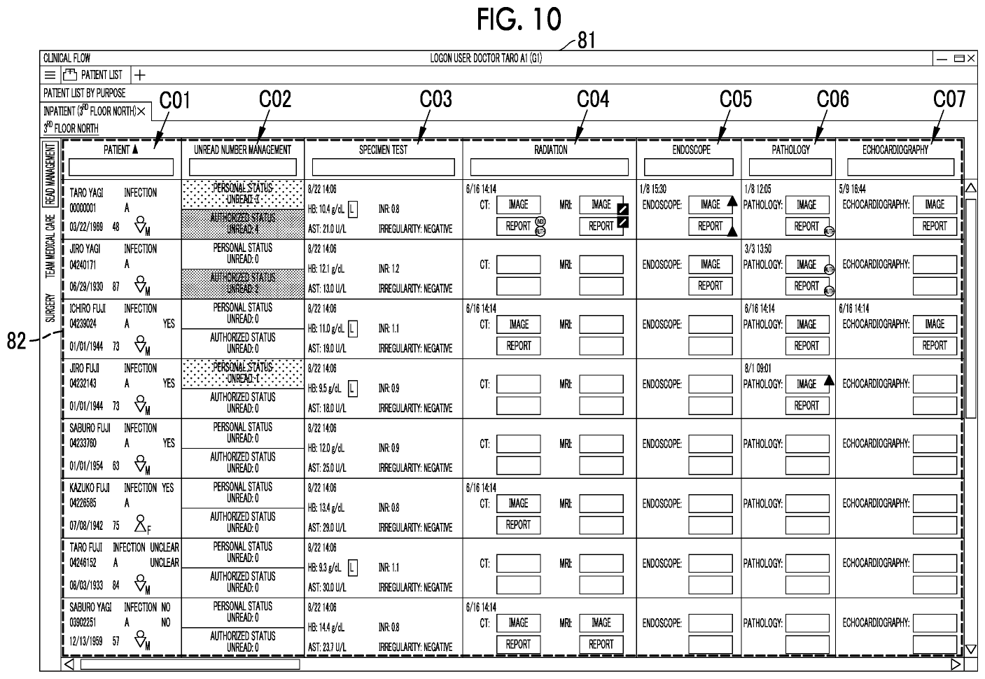

The initial screen 71 has, for example, three display fields of a schedule display field 72, a mail display field 73, and a list display field 74. The display contents of the schedule display field 72 and the mail display field 73 are generated by a gadget engine, which is a part of the operation program 39 of the client terminal 11, by obtaining information from the client terminal 11 or other devices or systems. Further, in the present embodiment, the list display field 74 displays at least a part of the clinical flow screen 81 shown in FIG. 10. Therefore, the display screen generation unit 62 generates the initial screen 71 including the schedule display field 72 and the mail display field 73 that do not include the contents of the clinical flow screen 81, and the list display field 74 that includes the contents of the clinical flow screen 81. The client terminal 11 uses a gadget engine to display the initial screen 71 supplemented with the contents of the schedule display field 72 and the mail display field 73 on the screen of the display unit 36.

In a case where all the contents to be displayed do not fit in the list display field 74, a scroll bar 78 and a scroll bar 79 for transitioning (so-called scrolling) the display contents of the list display field 74 are displayed in the list display field 74 or in the vicinity of the list display field 74. The scroll bar 78 is a GUI that is operated in a case where the display content of the list display field 74 is changed in the horizontal direction and a non-display portion is displayed. The scroll bar 79 is a GUI that is operated in a case where the display content of the list display field 74 is changed in the vertical direction and a non-display portion is displayed. The GUI control unit 41 performs such GUI display and control.

On the above initial screen 71, for example, in a case where a predetermined menu or the like is operated using a GUI such as a pointer (not shown), the request issuing unit 42 issues a distribution request for the display screen. The operation of issuing the distribution request for the display screen by the request issuing unit 42 based on the operation of the GUI or the like constitutes a display screen distribution request step. In the present embodiment, in order to display the entire clinical flow screen 81 in which a part of the list display field 74 is displayed, an operation for displaying the clinical flow screen 81 is executed. Thereby, the request issuing unit 42 issues a distribution request for the clinical flow screen 81.

In a case where the request issuing unit 42 issues a distribution request for the display screen, in the medical care support device 12, the request reception unit 61 receives the distribution request for the display screen, and the display screen generation unit 62 generates the display screen related to the distribution request for the display screen. The operation of the display screen generation unit 62 to generate the display screen related to the distribution request for the display screen constitutes a display screen generation step. In the present embodiment, the display screen generation unit 62 generates the clinical flow screen 81 by using the information related to the medical care process or the like appropriately acquired from the server group 13 or the like.

As described above, in a case where the display screen generation unit 62 generates the display screen related to the distribution request, before the generation of the display screen, at the same time as the generation of the display screen (in parallel with the generation of the display screen), or after the display screen is generated, the unread management unit 63 appropriately acquires each status of the main body status and the viewer-specific status of the medical care process to be displayed on the display screen from the server group 13 or the like. In the present embodiment, the unread management unit 63 acquires the statuses of “revised” and “important” from the server group 13 or the like in addition to the unread, read, or both of the medical care process as each status of the main body status and the viewer-specific status.

Then, the unread management unit 63 edits the display screen generated by the display screen generation unit 62, or causes the display screen generation unit 62 to further edit the display screen. Thereby, the unread management unit 63 displays each status of the main body status and the viewer-specific status on the display screen. In the present embodiment, the display screen generation unit 62 generates the clinical flow screen 81. Then, the unread management unit 63 acquires each status of the main body status and the viewer-specific status of the medical care process to be displayed on the clinical flow screen 81, and displays the status on the clinical flow screen 81. The operation of the unread management unit 63 to acquire each status of the main body status and the viewer-specific status of the medical care process constitutes a status acquisition step. Further, on the display screen generated by the display screen generation unit 62, the operation of the unread management unit 63 that displays each status of the main body status and the viewer-specific status of the medical care process in an identifiable manner constitutes a status display step.

After that, the GUI control unit 41 of the client terminal 11 receives the distribution of the display screen generated as described above, and the distributed screen is displayed on the screen of the display unit 36 instead of the initial screen 71, or is superimposed while leaving the initial screen 71 and displayed in another window or the like.

As shown in FIG. 10, the clinical flow screen 81 generated in the present embodiment comprises a clinical flow display field 82 having, for example, a patient column COT, an unread number management column C02, a biopsy column C03, a radiation column C04, an endoscope column C05, a pathology column C06, an echocardiography column C07, and the like. The patient column COT constitutes a list of a plurality of patients satisfying a predetermined condition. The predetermined condition is, for example, that the person in charge is a medical staff who is a login user, or that the person in charge is a group (medical department, etc.) to which the medical staff who is a login user belongs.

The unread number management column C02 is a collection of fields for displaying the number of unread items of the main body status and the viewer-specific status, and in the present embodiment, the viewer-specific status is displayed in the upper field, and the main body status is displayed in the lower field. Further, in the present embodiment, in a case where there is “unread” in the main body status, the background of the display field for the main body status is colored and highlighted. Similarly, in the present embodiment, in a case where there is “unread” in the viewer-specific status, the background of the display field for the viewer-specific status is colored and highlighted. Further, in the present embodiment, the background of the display field is colored in a darker color in the case where there is “unread” in the main body status than in the case where there is “unread” in the viewer-specific status. By doing so, it is possible to emphasize the presence of the unread, especially the presence of the unread of the main body status, and reduce the oversight.

The biopsy column C03, the radiation column C04, the endoscope column C05, the pathology column C06, the echocardiography column C07, and the like (columns other than the patient column COT and the unread number management column C02) each are a collection of fields for displaying the process or result of each examination or the like, which is the display name thereof. Each column of the biopsy column C03, the radiation column C04, the endoscope column C05, the pathology column C06, the echocardiography column C07, and the like (columns other than the patient column COT and the unread number management column C02) constitutes a medical care process column that displays the medical care process. Similar to the list display field 74 on the initial screen 71, the clinical flow display field 82 can display a non-display portion by a scroll operation or the like.

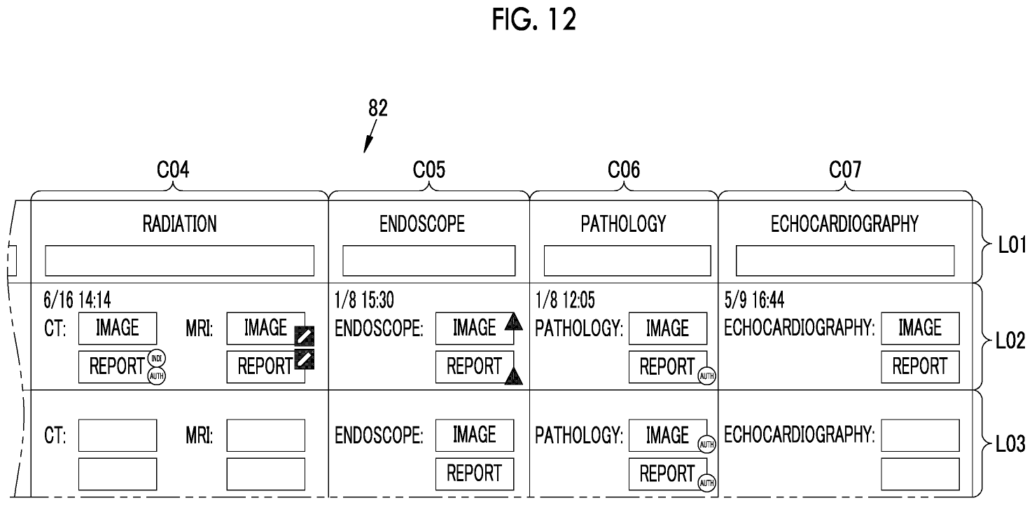

Further, as shown in FIGS. 11 and 12, the clinical flow display field 82 has an item display row L01 indicating display items of each column in the first row. By clicking the item name or the like in each field of the item display row L01 with a pointer or the like, the display contents can be sorted row by row according to descending order, ascending order, or other designated conditions. Sorting row by row means changing the display order while maintaining the correspondence in the row direction. For example, in a case where “patient” is clicked in the patient column C01 and the item display row L01, the request issuing unit 42 issues an edit request for the display screen. Then, the display screen generation unit 62 edits the clinical flow screen 81 according to a predetermined condition such as the order of the patient ID and provides the edited screen to the client terminal 11. Thereby, the display contents of the clinical flow display field 82 are sorted in the order of the patient ID and the like. Further, a mark (for example, a “▴” mark) indicating that the sorting has been performed is displayed on each item of the item display row L01 used as the sorting standard.

In the clinical flow display field 82, each row after the second row constitutes a so-called clinical flow in which the identification information of each patient and the medical care process of the patient are associated with each other. In FIGS. 11 and 12, the clinical flow of each of the two patients, “Taro Yagi” shown in row L02 and “Jiro Yagi” shown in row L03, is exemplified. In addition, the medical care process included in the clinical flow of each patient is managed and displayed for each item (for example, medical care data) included in each medical care process. For example, for radiographic imaging examination (radiation column C04), which is one of the medical care processes, the status such as an unread status is managed and displayed separately for the CT “image”, the CT “report”, the MRI “image”, and the MRI “report”. In the medical care process field of each patient (for example, the field of row L02 and column C04), in a case where the “image” or “report” is displayed, it indicates that the medical care data has already been acquired. Then, by not displaying the “image” or “report”, it indicates that the “image” or “report” has not been acquired or is not scheduled to be acquired (see, for example, the field of row L03 and column C04). The same applies to other medical care process fields.

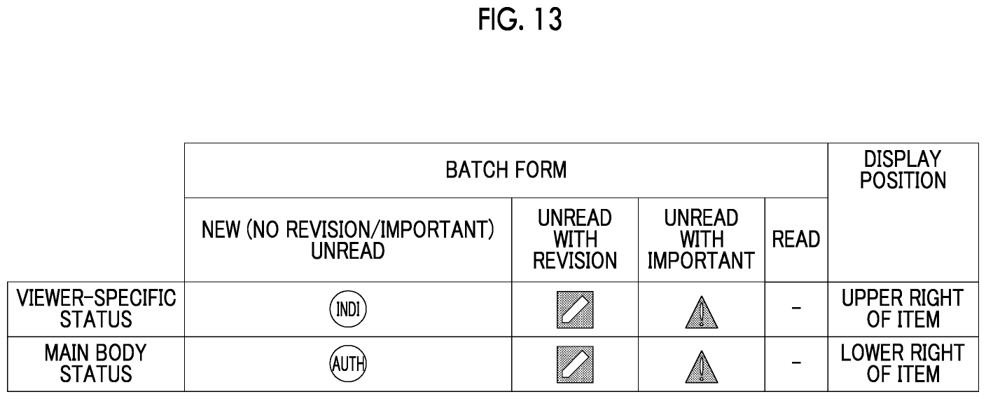

The main body status and the viewer-specific status are displayed with a “badge”, which is a mark indicating the content of the status, for each item of the medical care process. As shown in FIG. 13, the unread management unit 63 attaches, to a corresponding item, a “badge” each having a circular appearance in a case where a new item without “revision” and “important” is “unread”, a square appearance in a case where the “revised” item is “unread”, and a triangular appearance in a case where the item with “important” is unread. In addition, the unread management unit 63 does not attach a “badge” in a case where it is marked as read regardless of whether it has been “revised” or whether “important” has been attached. This makes it clear in a case where new items, revised items, and items with “important” are “unread”. Further, the unread management unit 63 attaches the above-mentioned “badge” to the upper right of the item in a case where the viewer-specific status is “unread”, and attaches the above-mentioned “badge” to the lower right of the item in a case where the main body status is “unread” (see FIG. 12). This makes it clear whether the “unread” status is the viewer-specific status or the main body status.

In the present embodiment, the form of the “badge” in the case of “unread” is changed between the viewer-specific status and the main body status. Specifically, the thickness of the border of the appearance of the “badge” is made different between the case where the viewer-specific status is “unread” and the case where the main body status is “unread”, and the border is thicker in the case where the main body status is “unread”. For new items, in the case where the viewer-specific status is “unread”, the characters inside the “badge” are set to “INDI” to indicate that the viewer himself/herself is “unread”, and in the case where the main body status is “unread, the characters inside the “badge” are set to “AUTH” to indicate that the authorized person (management target person in the present embodiment) who can change the main body status to the read is “unread”. Furthermore, for new items, the thickness of the characters inside the “badge” is thicker in the case where the main body status is “unread” than in the case where the viewer-specific status is “unread”. In addition, for the “revised” items and the items with “important”, the border of the mark inside the “badge” is thicker in the case where the main body status is “unread” than in the case where the viewer-specific status is “unread”. Thereby, regardless of the display position of the “badge”, it is possible to identify whether the “unread” status is the viewer-specific status or the main body status. Therefore, the position of the “badge” displayed in a case where the viewer-specific status is unread and the position of the “badge” displayed in a case where the main body status is unread can be set as a common position.

In FIG. 12, for example, the item of the CT report in the field of row L02 and column C04 is attached with a badge indicating that the viewer-specific status is unread and a badge indicating that the main body status is unread. Therefore, it is clear that the CT report of the patient (Taro Yagi) has not been read by either the viewer himself/herself or the management target person. In addition, the item of the MRI image in the same field is attached with a badge indicating that the revision has been made and the main body status is unread. Therefore, it is clear that the MRI image of the patient has been revised and that the viewer himself/herself has read it but the management target person has not read it. Further, the item of the MRI report in the same field is attached with a badge indicating that the revision has been made and the viewer-specific status is unread. Therefore, it is clear that the MRI report of the patient has been revised and that the viewer himself/herself has not read it but the management target person has read it.

In addition, the item of the image of the endoscope in the field of row L02 and column C05 is attached with a badge indicating that it is important and the viewer-specific status is unread. Therefore, it is clear that the image of the endoscope of the patient (Taro Yagi) is important, and that the viewer himself/herself has not read it but the management target person has read it. In addition, the item of the report of the endoscope in the same field is attached with a badge indicating that it is important and the main body status is unread. Therefore, it is clear that the report of the endoscope of the patient is important, and that the viewer himself/herself has read it but the management target person has not read it.

As described above, in the medical care support device 12, the unread management unit 63 manages the viewer-specific status and the main body status of the medical care process separately. Then, on the display screen such as the clinical flow screen 81, the viewer-specific status and the main body status are displayed in an identifiable manner. Therefore, on the display screen such as the clinical flow screen 81, the status such as an unread status between the viewer-specific status and the main body status of each medical care process is clear, so that the oversight of the unread medical care process can be reduced. In particular, it is easy to grasp the situation or the like that the management target person has not read it even though the viewer himself/herself has read it, which is effective in reducing the oversight that the management target person has not read it.

In the present embodiment, a configuration in which both the viewer-specific status and the main body status of each medical care process are displayed in an identifiable manner on a display screen such as the clinical flow screen 81 has been described as an example, but the present disclosure is not limited thereto. A configuration may be used in which only one of the viewer-specific status and the main body status is displayed on the display screen. Further, a configuration may be used in which whether to display any of the viewer-specific status and the main body status can be selected. Of course, a configuration may be used in which the presence or absence of display can be selected for each of the viewer-specific status and the main body status.

### Second Embodiment

In the first embodiment, the viewer-specific status and the main body status are displayed in an identifiable manner on the clinical flow screen 81. However, as shown in FIG. 14, in a case where the display screen generation unit 62 generates or edits the timeline screen 201, similar to the clinical flow screen 81 of the first embodiment, the unread management unit 63 can display the viewer-specific status and the main body status in an identifiable manner on the timeline screen 201.

The timeline screen 201 displays a time element such as a date and time and a medical care process in association with each other. For example, in FIG. 14, it is displayed that the examination by CT was performed in the field of “19/06/12” (Jun. 12, 2019). In this case, the unread management unit 63 displays the viewer-specific status and the main body status in an identifiable manner on the timeline screen 201 by attaching, for example, a badge (see FIGS. 12 and 13) similar to the clinical flow screen 81 of the first embodiment to the display of an “image” indicating the CT examination image or a “report” indicating the report. In FIG. 14, the CT report (“report”) is attached with a badge indicating that there is a revision and that the viewer-specific status is unread.

As described above, in a case where the viewer-specific status and the main body status are displayed in an identifiable manner on the timeline screen 201, it is possible to grasp the date and time when the medical care process was performed or the date and time scheduled to be performed in the future in association with the viewer-specific status and the main body status. For example, in a case where the same type of medical care process is performed a plurality of times, it is useful to reduce oversights such as unread by being able to distinguish the medical care process of each time and grasp the viewer-specific status and the main body status.

### Third Embodiment

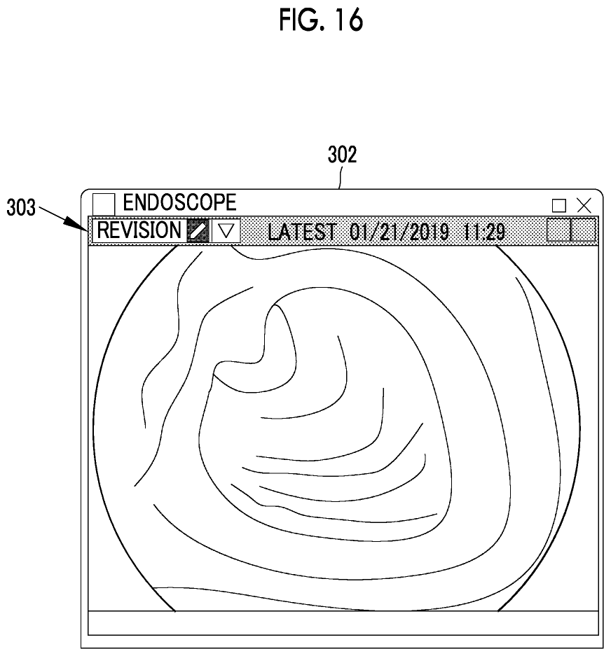

As shown in FIG. 15, in a case where the display screen generation unit 62 generates or edits the layout screen 301, similar to the clinical flow screen 81 of the first embodiment, the unread management unit 63 can display the viewer-specific status and the main body status in an identifiable manner on the layout screen 301. In FIG. 15, the unread management unit 63 attaches a badge indicating that there is a revision and the main body status is unread, together with the characters “revised” to the examination image of the endoscope in the endoscopic image field 302 that constitutes a part of the layout screen 301. More specifically, as shown in FIG. 16, the endoscopic image field 302 is provided with a status display field 303 for displaying the viewer-specific status and the main body status in an identifiable manner. Then, in the status display field 303, together with the characters “revised”, a badge indicating that there is a revision and the main body status is unread is attached. As described above, in a case where the viewer-specific status and the main body status are displayed in an identifiable manner on the layout screen 301, it is useful for reducing oversights such as unread because it is possible to grasp the viewer-specific status and the main body status while listing the outline of the medical care process of a patient.

### Fourth Embodiment

As shown in FIG. 17, in a case where the display screen generation unit 62 generates or edits the unread data list screen 401, similar to the clinical flow screen 81 of the first embodiment, the unread management unit 63 can display the viewer-specific status and the main body status in a list in an identifiable manner on the unread data list screen 401. In FIG. 17, medical care processes whose status is unread are extracted, and the patient name, data type, examination/revision date, badge, and status type are displayed in a list for each medical care process. By displaying a list of medical care processes whose status is unread in this way, it is possible to more reliably reduce oversights such as unread. Although the medical care processes that have been unread are displayed in a list, the medical care processes that have been read may be displayed in a list. Of course, all medical care processes may be displayed in a list regardless of whether the medical care processes have been unread or read.

### Fifth Embodiment

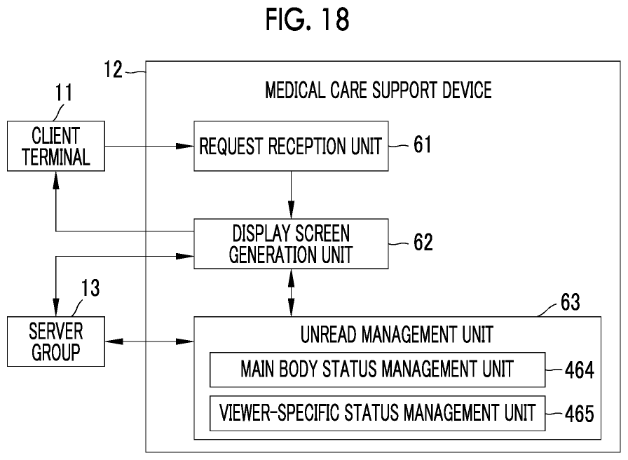

In the first embodiment or the like, a case where the unread management unit 63 is provided with the main body status management table 64 and the viewer-specific status management table 65, and the unread management unit 63 manages the main body status using the main body status management table 64 and manages the viewer-specific status using the viewer-specific status management table 65 has been described as an example, but the present disclosure is not limited thereto. As shown in FIG. 18, the unread management unit 63 may be provided with a main body status management unit 464 and a viewer-specific status management unit 465, and the main body status management unit 464 may manage the main body status, and the viewer-specific status management unit 465 may manage the viewer-specific status.

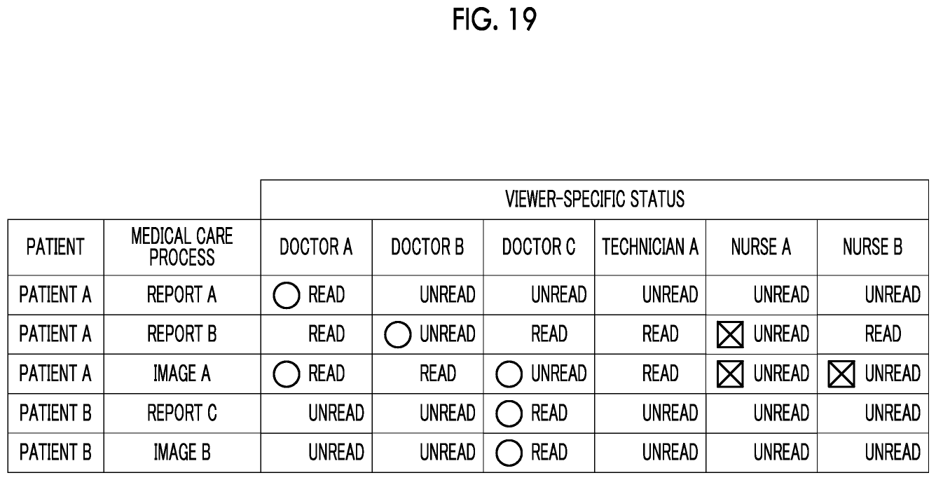

As shown in FIG. 19, the viewer-specific status management unit 465 manages each medical care process and the status of each viewer (medical staff) (which is the viewer-specific status, and in the present embodiment, whether the target medical care process is unread or read) in association with each other. In the example of FIG. 19, the authority of each viewer is indicated by the indicator on the left side of the viewer-specific status. Specifically, a circular indicator is attached to a management target person, and a square indicator is attached to a non-read target person (a person who does not have viewing authority). In addition, none of the indicators is attached to non-management target persons (persons who have viewing authority (read target persons) but are not management target persons).

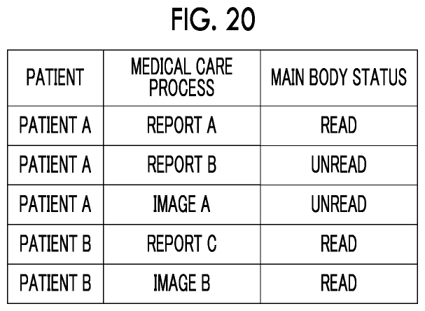

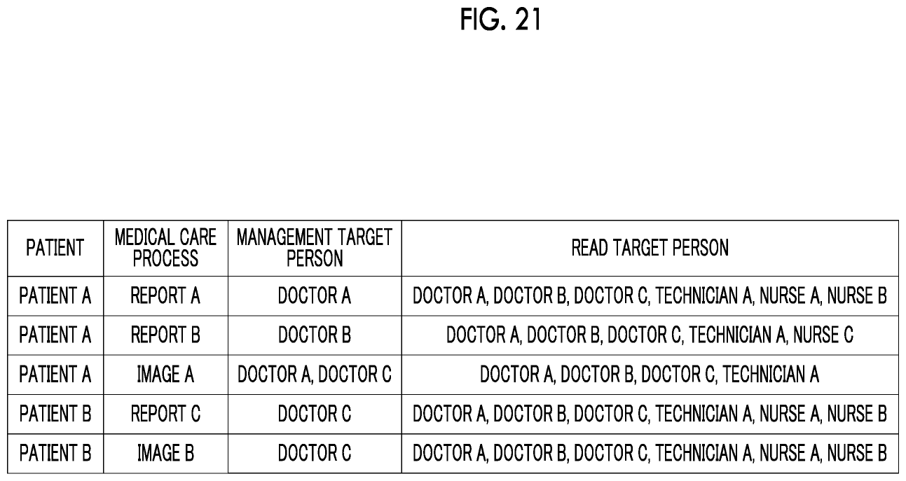

The main body status management unit 464 manages the main body status of each medical care process by accessing the viewer-specific status management unit 465 as necessary, referring to the viewer-specific status and the authority of each viewer. In the example of FIG. 19, a report A of a patient A has been read by a doctor A who is the management target person. In addition, a report C and an image B of a patient B have also been read by a doctor C who is the management target person. Therefore, for these, the main body status management unit 464 manages the main body status as read as shown in FIG. 20. On the other hand, in the example of FIG. 19, a report B of the patient A is unread by a doctor B who is the management target person, and an image A of the patient A is read by the doctor A who is the management target person, but is unread by the doctor C who is also the management target person. Therefore, for these, as shown in FIG. 20, the main body status management unit 464 manages the main body status as unread. As shown in FIG. 21, a correspondence table may be provided in which each medical care process is associated with an authorized person (in the example of FIG. 21, a management target person and a read target person), and the main body status management unit 464 may refer to the correspondence table and manage the main body status.

In the first embodiment or the like, a display screen such as the clinical flow screen 81 is generated and displayed via the initial screen 71, but the display screen such as the clinical flow screen 81 can be indirectly generated from another device or system that is linked with the medical care support device 12. For example, since the electronic medical record server 21 can be operated directly from the client terminal 11, the clinical flow screen 81 can be called in the process of operating the electronic medical record server 21.

On the display screen such as the clinical flow screen 81, the timeline screen 201, the layout screen 301, or the unread data list screen 401 of the first embodiment or the like, patients, medical care processes, items included in the medical care process, or the like can be randomly searched, sorted, or selected.

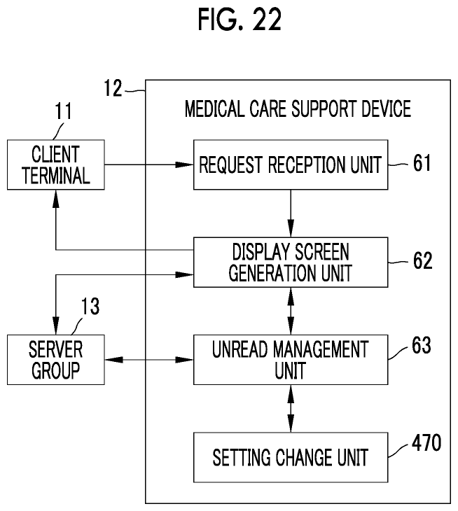

As shown in FIG. 22, the medical care support device 12 of the first embodiment or the like preferably comprises a setting change unit 470 for changing the setting of the viewer and the type of viewer. The setting change unit 470 receives a change operation for changing the contents of the viewer-specific status management table 65 (see FIG. 8), and changes the contents of the viewer-specific status management table 65, for example, according to this change operation. The change operation is input from the operation unit 37 of the client terminal 11 via the GUI, for example. It is preferable that the change operation is performed on the display screen in a state where the viewer-specific status management table 65 is displayed on the display screen. Specifically, it is preferable that the change operation such as changing the contents of each field, adding a row, deleting a row, adding a column, and deleting a column is performed on the displayed viewer-specific status management table 65. That is, it is preferable to change the settings of the viewer and the type of viewer by displaying a correspondence list showing a correspondence between the medical care process and the read target person (in the case of this example, the viewer-specific status management table 65) and reflecting a change operation performed on the correspondence list. By changing the contents of the viewer-specific status management table 65, for example, it is possible to add a read target person, add, delete, or change a management target person, and change a status (whether the status is read or unread). The setting change unit 470 is one function of the operation program 59 (see FIG. 5), and the CPU 51 and the memory 52 cooperate to function as the setting change unit 470. The above-mentioned change may be made, for example, by selecting a plurality of fields (for example, all the statuses related to the doctor A) from the viewer-specific status management table 65 and collectively performing the selected plurality of fields. Further, the setting change unit 470 may not only change the set viewer and the set type of viewer, but also newly set viewers and the type of viewers (newly set (newly register) medical care processes, viewers, and the type of viewers in the viewer-specific status management table 65) for the medical care processes in which viewers and the type of viewers are not set (the medical care processes that are not set (unregistered) in the viewer-specific status management table 65).

The setting change unit 470 described above can perform a process of taking over the authority of oneself or another person regarding viewing to another person who does not have this authority (hereinafter referred to as a takeover process). For example, in a case where the doctor who is the management target person cannot mark the medical care process as read due to a long business trip, etc., the management target person can be changed to another person (a takeover person) by performing the takeover process. Of course, it is possible to perform the takeover process on the authority of a person other than the management target person (for example, a read-out person (non-management target person) other than the management target person). It is also possible to select a plurality of medical care processes and collectively perform the takeover process for these plurality of medical care processes.

In addition, as the management target person, for example, it is preferable that a doctor or the like (a first viewer) who is allowed to deal with the medical care process alone is set as a new or takeover target. However, for example, in some cases, a resident or the like (a second viewer) who is not allowed to deal with the medical care process alone and is obliged to deal with the medical care process under the supervision of an instructor is set as a new management target person. Therefore, in a case where a correspondence table is provided in which a second viewer of the resident, etc. is associated with a first viewer of the instructor, etc. who guides the resident and the second viewer is set as the management target person, the main body status may not be marked as read unless the first viewer associated with the second viewer has read the medical care process. Specifically, in a case where the second viewer is set as the management target person, it is conceivable that the unread management unit 63 maintains the main body status as unread in the case where only the status of the second viewer (viewer-specific status) is marked as read, and sets the main body status as read in the case where the status (viewer-specific status) of both the second viewer and the first viewer associated with the second viewer is marked as read. Further, in a case where the second viewer is set as the management target person, the setting change unit 470 may set the first viewer associated with the second viewer as the management target person in synchronization with this, and the unread management unit 63 may maintain the main body status as unread until the status (viewer-specific status) of all the management target persons is marked as read.

Further, the setting change unit 470 can perform a process (hereinafter referred to as a proxy read process) in which the non-read target person or the like changes the main body status to read on behalf of the management target person. That is, in a case where a person such as a non-read target person (hereinafter referred to as a read proxy) who has been requested and/or approved for the proxy read process from the management target person views, the setting change unit 470 can change the viewer-specific status of the management target person of the target medical care process to read. For example, regarding the report B of FIG. 8, the doctor A has not read it, but the doctor C who has been requested to perform the proxy read process views the report B, and thereby, as shown in FIG. 23, the viewer-specific status of the report B of the doctor A is changed to read, and the doctor C is registered as the read performer. Then, the viewer-specific status of the doctor A who is the management target person of the report B is changed to read, and thereby, this result is reflected in the main body status and the main body status is also marked as read.

In addition, the doctor C who has been requested to perform the proxy read process views the report B, and thereby, as shown in FIG. 24, the management target person may shift from the doctor A to the doctor C, and the unread state of the doctor A may be maintained. In this case as well, the doctor C who has been requested to perform the proxy read process views the report B, so that the viewer-specific status of the doctor C who is the management target person is marked as read, and thereby, this is reflected in the main body status and the main body status is also marked as read.

On the other hand, in a case where the doctor C views the report B even though he/she does not have the authority to perform the proxy read process, such as not being requested to perform the proxy read process or not received approval for the proxy read process, as shown in FIG. 25, the viewer-specific status or the like regarding the doctor A is not changed, and only the fact that the doctor C has viewed the report C is simply registered. In this case, since the doctor A who is the management target person remains unread, the main body status is also maintained as unread.

As described above, it is preferable that the setting change unit 470 changes the contents of the viewer-specific status management table 65 by adding a person (viewer) of a request destination who has made such a request to the read target person in a case of making various requests, such as a case of requesting the proxy read process, a case of requesting the viewing or the like of the medical care process scheduled to be executed in the future, or a case of requesting the viewing or the like of the medical care process which has been executed but is in the unread state, and furthermore, a case of requesting the medical care (execution of the medical care process).

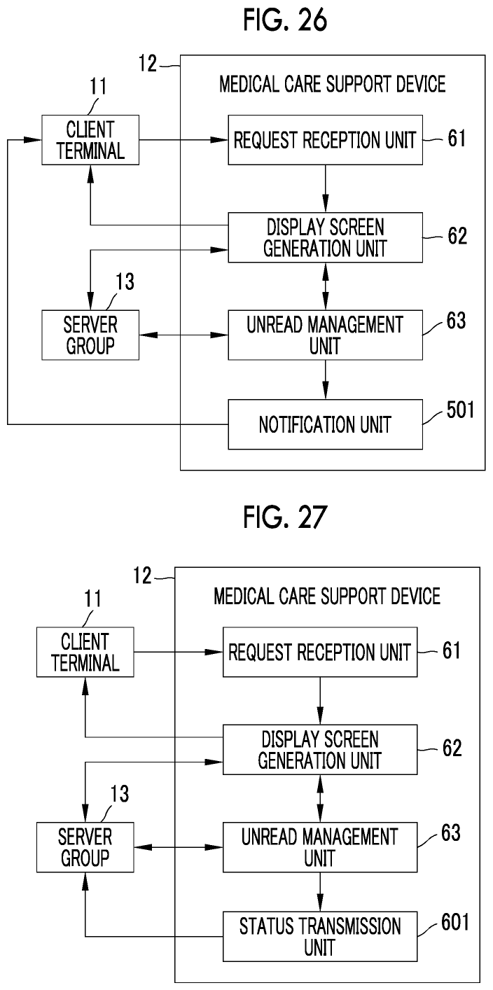

Further, in the case of making various requests as described above, as shown in FIG. 26, it is preferable to provide a notification unit 501 and send a comment together with the request. The notification unit 501 receives the input of a comment from the requester through the request reception unit 61, and sends (notifies) the comment to the request destination such as the proxy read process by attaching the comment to the request or the like. The notification unit 501 is one function of the operation program 59 (see FIG. 5), and the CPU 51 and the memory 52 cooperate to function as the notification unit 501. By notifying the comment in this way, the medical staff at the request destination can easily understand the content of the request and/or the intention of the request, which is convenient.

The comment is not limited to the form of being attached to a request from a request source and being sent to the client terminal 11 of the request destination, and can be appropriately sent in other forms. For example, in a case where a proxy read process is requested, a comment may be sent to the unread management unit 63 by attaching the comment to the request from the request source. In this case, it is preferable that the unread management unit 63 makes the sent comment viewable, for example, by linking and storing the comment in the status field of the viewer-specific status management table and referring to the link destination. By doing so, it is convenient because it is possible to confirm the reason why the proxy read process was performed or the like by referring to the comment later. Of course, in a case where there is a request for a proxy read process or the like, a comment may be sent to both the client terminal 11 and the unread management unit 63 of the request destination.

Further, the input of the comment from the client terminal 11 of the request destination that has performed the proxy read process or the like in response to the request may be received, and the received comment may be sent to the client terminal 11 of the request source. Of course, in a case where a process such as a change in status, viewer, type of viewer, or the like, or new setting (registration) is performed without requesting a proxy read process, etc., a comment may be input and send for this process. Further, in a case where a medical care (medical care process) is executed, a comment may be input and sent regarding the process and result of the medical care (medical care process). Further, the presence or absence of such a comment and/or the content of the comment may be displayed on the display screen generated by the display screen generation unit 62 in the same manner as the unread/read status described above. In this case, in a case where a badge indicating the presence or absence of a comment is displayed on or around the corresponding medical care process on the display screen and this badge is selected by clicking or the like, it is conceivable to display the content of the comment by opening a new window, for example.

Furthermore, with a request for a proxy read process, a process such as changing the status, viewer, type of viewer, new setting (registration), or execution of medical care (medical care process), accessory information other than the above-mentioned comments may be input and sent. The accessory information is, for example, information indicating that the explanation to the patient has been completed, information indicating that the examination instruction to the examination department or the like has been completed, and the like. It is conceivable that these pieces of accessory information are displayed on or around the medical care process, for example, in the form of a check box (the form in which a check mark is displayed in the check box by clicking or the like in the case of being completed).

Further, in the first embodiment or the like, in a case where the medical care process for which a user himself/herself is a management target person is viewed and the viewer-specific status is marked as read, this is reflected in the main body status and the main body status is also marked as read. However, the medical care support device 12 may be operated in another operation mode (hereinafter referred to as a special operation mode) different from such an operation mode (hereinafter referred to as a normal mode). Specifically, in a case where the medical care support device 12 is operating in the special operation mode, the viewer-specific status may not be marked as read (naturally, the main body status is also maintained as unread) even though the medical care process for which a user himself/herself is a management target person is viewed, or the main body status may be maintained as unread without being reflected in the main body status even though the viewer-specific status is marked as read by viewing the medical care process for which a user himself/herself is a management target person. Of course, in the case of operating in the special operation mode, the viewer-specific status may be maintained as unread even though the medical care process for which a user himself/herself is a non-management target person (a person who is a read target person but not a management target person) is viewed. In this way, in a case where the normal mode and the special operation mode are provided, the user is allowed to select in which mode the medical care support device 12 is operated in the case of logging in to the medical care support system 10 (or the medical care support device 12) or the like via a GUI or the like. Furthermore, regardless of user's own viewing authority (whether it is a management target person (read target person), a non-management target person (read target person), or a non-read target person), the medical care support device 12 may be operated in an operation mode in which the status of himself/herself and/or another person can be changed (hereinafter referred to as a status change mode). In the status change mode, the unread management unit 63 changes the contents of the main body status management table 64 and the viewer-specific status management table 65 according to the instruction from the user input via the GUI or the like.

As shown in FIG. 27, the medical care support device 12 of the first embodiment or the like preferably comprises a status transmission unit 601 that transmits the main body status and/or the viewer-specific status to a medical care support device (for example, a PACS server (image server 22) or the like) different from the medical care support device 12, such as a server constituting a PACS, or another medical care support system different from the medical care support system 10. The status transmission unit 601 periodically transmits, to another medical care support device or the like, the status of the type requested in a case where there is a transmission request from another medical care support device or the like, the status changed in a case where the main body status and/or the viewer-specific status is changed or the main body status and/or the viewer-specific status. In this way, by transmitting the main body status and/or the viewer-specific status to another medical care support device or the like, it is convenient because the oversight can be confirmed by another medical care support device or the like.

The medical care support device 12 of the first embodiment or the like can distribute a display screen generated and/or edited by the display screen generation unit 62 (for example, a display screen in which the main body status and/or the viewer-specific status are displayed in an identifiable manner) to another medical care support device (for example, a PACS server (image server 22) or the like) different from the medical care support device 12, such as a server constituting a PACS, or another medical care support system different from the medical care support system 10. In this case, the display screen generation unit 62 distributes the display screen to another medical care support device or the like different from the medical care support device 12, as in the case of distributing the display screen to the client terminal 11. The display screen generation unit 62 distributes the display screen according to the distribution request for the display screen input from another medical care support device or the like. In this way, even in a case where the display screen is distributed to another medical care support device or the like, it is convenient because the oversight can be confirmed by the other medical care support device or the like.

In addition, the medical care support device 12 of the first embodiment or the like can transmit and receive various requests, comments, accessory information, and the like described above to and from another medical care support device (for example, a PACS server (image server 22) or the like) different from the medical care support device 12, such as a server constituting a PACS, or another medical care support system different from the medical care support system 10.

The medical care support system 10 according to the first embodiment or the like comprises: the display screen generation unit 62 that generates a display screen for displaying patient identification information and a medical care process in association with each other for each of a plurality of patients; an unread management unit 63 that displays, on the display screen, a status including information indicating the medical care process being unread, read, or both of them, and manages a viewer-specific status separately from a main body status that is the status of the medical care process, the viewer-specific status being the status of each viewer who views the display screen; and a status change unit (the unread management unit 63) that changes the main body status by referring to the viewer-specific status and reflecting a status of a specific type of viewer among a plurality of types of viewers in the main body status. The unread management unit 63 displays at least one of the viewer-specific status or the main body status in an identifiable manner on the display screen.

Further, the operation program 59 of the medical care support device 12 according to the first embodiment or the like causes the CPU 51 or the CPU 51 and the memory 52 to function as the display screen generation unit 62 that generates a display screen for displaying patient identification information and a medical care process in association with each other for each of a plurality of patients; an unread management unit 63 that displays, on the display screen, a status including information indicating the medical care process being unread, read, or both of them, and manages a viewer-specific status separately from a main body status that is the status of the medical care process, the viewer-specific status being the status of each viewer who views the display screen; and a status change unit (the unread management unit 63) that changes the main body status by referring to the viewer-specific status and reflecting a status of a specific type of viewer among a plurality of types of viewers in the main body status. The unread management unit 63 displays at least one of the viewer-specific status or the main body status in an identifiable manner on the display screen.

That is, the medical care support device 12 according to the first embodiment or the like causes a processor to execute: generating a display screen for displaying patient identification information and a medical care process in association with each other for each of a plurality of patients; displaying, on the display screen, a status including information indicating the medical care process being unread, read, or both of them, and managing a viewer-specific status separately from a main body status that is the status of the medical care process, the viewer-specific status being the status of each viewer who views the display screen; changing the main body status by referring to the viewer-specific status and reflecting a status of a specific type of viewer among a plurality of types of viewers in the main body status; and displaying at least one of the viewer-specific status or the main body status in an identifiable manner on the display screen.

An operation method of the medical care support device 12 according to the first embodiment is an operation method of the medical care support device 12 comprising the display screen generation unit 62 that generates a display screen for displaying patient identification information and a medical care process in association with each other for each of a plurality of patients, an unread management unit 63 that displays, on the display screen, a status including information indicating the medical care process being unread, read, or both of them, and manages a viewer-specific status separately from a main body status that is the status of the medical care process, the viewer-specific status being the status of each viewer who views the display screen, and a status change unit (the unread management unit 63) that changes the main body status by referring to the viewer-specific status and reflecting a status of a specific type of viewer among a plurality of types of viewers in the main body status. The operation method comprises a status display step of displaying, by the unread management unit 63, at least one of the viewer-specific status or the main body status in an identifiable manner on the display screen.

An operation method of the medical care support system 10 according to the first embodiment or the like is an operation method of the medical care support system 10 comprising the display screen generation unit 62 that generates a display screen for displaying patient identification information and a medical care process in association with each other for each of a plurality of patients, an unread management unit 63 that displays, on the display screen, a status including information indicating the medical care process being unread, read, or both of them, and manages a viewer-specific status separately from a main body status that is the status of the medical care process, the viewer-specific status being the status of each viewer who views the display screen, and a status change unit (the unread management unit 63) that changes the main body status by referring to the viewer-specific status and reflecting a status of a specific type of viewer among a plurality of types of viewers in the main body status. The operation method comprises a status display step of displaying, by the unread management unit 63, at least one of the viewer-specific status or the main body status in an identifiable manner on the display screen.

In the first embodiment or the like, hardware structures of the processing units that execute various processes such as the GUI control unit 41, the request issuing unit 42, the request reception unit 61, the display screen generation unit 62, the unread management unit 63, the setting change unit 470, the notification unit 501, and the status transmission unit 601 are various processors as shown below. The various processors include a central processing unit (CPU) that is a general-purpose processor that functions as various processing units by executing software (program), a programmable logic device (PLD) that is a processor whose circuit configuration can be changed after manufacture, such as field programmable gate array (FPGA), a dedicated electrical circuit that is a processor having a circuit configuration designed exclusively for executing various types of processing, and the like.

One processing unit may be configured by one of various processors, or may be configured by a combination of two or more processors of the same or different kinds (for example, a combination of a plurality of FPGAs or a combination of a CPU and an FPGA). In addition, a plurality of processing units may be configured by one processor. As an example of configuring a plurality of processing units by one processor, first, as represented by a computer, such as a client or a server, there is a form in which one processor is configured by a combination of one or more CPUs and software and this processor functions as a plurality of processing units. Second, as represented by a system on chip (SoC) or the like, there is a form of using a processor for realizing the function of the entire system including a plurality of processing units with one integrated circuit (IC) chip. Thus, various processing units are configured by using one or more of the above-described various processors as hardware structures.

More specifically, the hardware structure of these various processors is an electrical circuit (circuitry) in the form of a combination of circuit elements, such as semiconductor elements.

### EXPLANATION OF REFERENCES

10: medical care support system
11: client terminal
12: medical care support device
13: server group
14: network
21: electronic medical record server
21A: medical record database
22: image server
22A: image database
23: report server
23A: report database
31: CPU
32: memory
33: storage
34: communication unit
35: connection circuit
36: display unit
37: operation unit
39: operation program
41: GUI control unit
42: request issuing unit
51: CPU
52: memory
53: storage
54: communication unit
55: connection circuit
59: operation program
61: request reception unit
62: display screen generation unit
63: unread management unit (status change unit)
64: main body status management table
65: viewer-specific status management table
71: initial screen
72: schedule display field
73: mail display field
74: list display field
78: scroll bar
79: scroll bar
81: clinical flow screen
82: clinical flow display field
201: timeline screen
301: layout screen
302: endoscopic image field
303: status display field
401: unread data list screen
464: main body status management unit
465: viewer-specific status management unit
470: setting change unit
501: notification unit
601: status transmission unit
A1: doctor
A2: doctor
B1: doctor
N1: technician
G1, G2, G19: group
C01: patient column
C02: unread number management column
C03: biopsy column
C04: radiation column
C05: endoscope column
C06: pathology column
C07: echocardiography column
L01, L02, L03: item display row

What is claimed is:

1. A medical care support device comprising
a processor configured to:
generate a display screen for displaying patient identification information and a medical care process in association with each other for each of a plurality of patients;
display, on the display screen, a status including information indicating the medical care process being unread, read, or both of them, and manage a viewer-specific status separately from a main body status that is the status of the medical care process, the viewer-specific status being the status of each viewer who views the display screen;
change the main body status by referring to the viewer-specific status and reflecting a status of a specific type of viewer among a plurality of types of viewers in the main body status; and
display at least one of the viewer-specific status or the main body status in an identifiable manner on the display screen.

2. The medical care support device according to claim 1, wherein the processor is configured to display any of the viewer-specific status or the main body status in a selectable manner on the display screen.

3. The medical care support device according to claim 1, wherein the processor is configured to display both the viewer-specific status and the main body status on the display screen.

4. The medical care support device according to claim 1,
wherein the viewer is classified into a read target person and a non-read target person other than the read target person,
the read target person is classified into a management target person who is the specific type of viewer and a non-management target person other than the management target person, and
in a case where the status of the management target person is changed from unread to read, the main body status is changed from unread to read by reflecting the changed status.

5. The medical care support device according to claim 4, wherein, in a case where a plurality of the management target persons are set and all the statuses of the plurality of management target persons are changed from unread to read, the main body status is changed from unread to read by reflecting the changed statuses.

6. The medical care support device according to claim 4,
wherein a correspondence table in which a first viewer who is allowed to deal with the medical care process alone and a second viewer who is prohibited from dealing with the medical care process alone are associated with each other is referred to, and
in a case where the second viewer is set as the management target person, the first viewer associated with the second viewer is set as the management target person together with the second viewer.

7. The medical care support device according to claim 4, wherein the processor is configured to display the viewer-specific status of the read target person in an identifiable manner on the display screen.

8. The medical care support device according to claim 1, wherein the processor is configured to change settings of the viewer and the type of viewer.

9. The medical care support device according to claim 4, wherein the processor is configured to approve, in a case where the management target person is changed, a change to the management target person before the change, and change, in a case where the change is approved, the management target person.

10. The medical care support device according to claim 9, wherein, in a case where the change is not approved, the viewer whose change is not approved is set as the non-management target person.

11. The medical care support device according to claim 4, wherein the processor is configured to set a viewer of a request destination who has been requested to perform the medical care process as the read target person.

12. The medical care support device according to claim 11, wherein the processor is configured to receive an input of a comment to the request destination and notify a designated request destination of the request and the comment.

13. The medical care support device according to claim 4, wherein the processor is configured to change the settings of the viewer and the type of viewer by displaying a correspondence list showing a correspondence between the medical care process and the read target person and reflecting a change operation performed on the correspondence list.

14. The medical care support device according to claim 1, wherein the processor is configured to transmit the viewer-specific status and the main body status to another medical care support device different from the medical care support device.

15. The medical care support device according to claim 1, wherein the processor is configured to transmit the display screen to another medical care support device different from the medical care support device.

16. A medical care support method comprising:
generating a display screen for displaying patient identification information and a medical care process in association with each other for each of a plurality of patients;
displaying, on the display screen, a status including information indicating the medical care process being unread, read, or both of them, and managing a viewer-specific status separately from a main body status that is the status of the medical care process, the viewer-specific status being the status of each viewer who views the display screen;
changing the main body status by referring to the viewer-specific status and reflecting a status of a specific type of viewer among a plurality of types of viewers in the main body status; and
displaying at least one of the viewer-specific status or the main body status in an identifiable manner on the display screen.

17. A non-transitory computer readable recording medium storing a medical care support program, the medical care support program causing a computer to execute:
generating a display screen for displaying patient identification information and a medical care process in association with each other for each of a plurality of patients;
displaying, on the display screen, a status including information indicating the medical care process being unread, read, or both of them, and managing a viewer-specific status separately from a main body status that is the status of the medical care process, the viewer-specific status being the status of each viewer who views the display screen;
changing the main body status by referring to the viewer-specific status and reflecting a status of a specific type of viewer among a plurality of types of viewers in the main body status; and
displaying at least one of the viewer-specific status or the main body status in an identifiable manner on the display screen.
